(19) United States
(12) Patent Application Publication
(10) Pub. No.: US 2010/0234309 A1
(43) Pub. Date: Sep. 16, 2010

(54) Dendrimer Compositions

(75) Inventors: Christopher Cooper, Sterling, MA (US); Pradeep K. Dhal, Westford, MA (US); Rayomand H. Gimi, Chelmsford, MA (US); David J. Harris, Lexington, MA (US); Stephen Randall Holmes-Farley, Arlington, MA (US); Chad C. Huval, Somerville, MA (US); Edward Lee, Sudbury, MA (US)

Correspondence Address: GENZYME CORPORATION;LEGAL DEPARTMENT, 15 PLEASANT ST CONNECTOR, FRAMINGHAM, MA, 01701-9322, US

(73) Assignee: Genzyme Corporation, Cambridge, MA (US)

(21) Appl. No.: 12/310,514

(22) PCT Filed: Aug. 31, 2007
(86) PCT No.: PCT/US07/19201

§ 371 Date: Jun. 4, 2010

Related U.S. Application Data
(60) Provisional application No. 60/841,566, filed on Sep. 1, 2006.
(60) Provisional application No. 60/849,434, filed on Oct. 5, 2006.
(60) Provisional application No. 60/853,440, filed on Oct. 23, 2006.

(51) Int. Cl. A61K 31/7004; A61P 7/00; A61P 39/00; A61K 31/13

(52) U.S. Cl. 514/23; 514/668

(57) ABSTRACT

Amine compounds, amine polymers, crosslinked amine polymers and pharmaceutical compositions comprising the same may include polyhydroxy-containing cores that may be substituted with amine groups and may be used to treat hyperphosphatemia or to remove ions from the gastrointestinal tract of animals, including humans.

### FIELD OF THE INVENTION

This invention relates to amine polymers for binding target ions, and more specifically relates to pharmaceutically acceptable compositions, amine dendrimers, amine polymers or residues thereof for binding target ions.

### BACKGROUND OF THE INVENTION

Hyperphosphatemia frequently accompanies diseases associated with inadequate renal function such as end stage renal disease (ESRD), hyperparathyroidism, and certain other medical conditions. The condition, especially if present over extended periods of time, leads to severe abnormalities in calcium and phosphorus metabolism and can be manifested by aberrant calcification in joints, lungs, and eyes.

Therapeutic efforts to reduce serum phosphate include dialysis, reduction in dietary phosphate, and oral administration of insoluble phosphate binders to reduce gastrointestinal absorption. Many such treatments have a variety of unwanted side effects and/or have less than optimal phosphate binding properties, including potency and efficacy. Accordingly, there is a need for compositions and treatments with good phosphate-binding properties and good side effect profiles.

### BRIEF SUMMARY OF THE INVENTION

In one aspect, the present invention relates to amine compounds, amine polymers and/or pharmaceutical compositions comprising, at least in part, amine compounds (including amine dendrimers) or residues thereof comprising substituted polyhydroxy cores, where one or more of the hydroxyl groups on the core are substituted via an ether linkage, or to form one or more ethers, with one or more amine moieties. The amine compounds can be crosslinked to form amine polymers. Compositions can comprise one or more amine compounds and/or amine polymers or residues thereof. Several embodiments of the invention, including this aspect of the invention, are described in further detail as follows. Generally, each of these embodiments can be used in various and specific combinations, and with other aspects and embodiments unless otherwise stated herein.

In addition to the amine compounds and amine polymers of the present invention as described herein, other forms of the amine polymers and amine compounds are within the scope of the invention including pharmaceutically acceptable salts, solvates, hydrates, prodrugs, polymorphs, clathrates, and isotopic variants and mixtures thereof of the amine compounds and/or amine polymers.

In addition, amine compounds and amine polymers of the invention may have optical centers, chiral centers or double bonds and the amine compounds and amine polymers of the present invention include all of the isomeric forms of these compounds and polymers, including optically pure forms, racemates, diastereomers, enantiomers, tautomers and/or mixtures thereof.

In a first embodiment, the invention is, consists essentially of, or comprises an amine compound or residue thereof or an amine polymer that comprises at least one amine compound or residue thereof, where the amine compound is represented by Formula I, as follows:

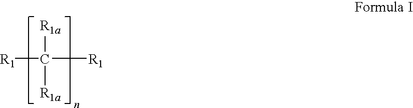

wherein n independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₁ independently represents a hydrogen radical, a hydroxyl radical or —OR₃; R₁ₐ independently represents R₁, —R₂OH or —R₂OR₃; with the proviso that the amine compound includes at least one moiety represented by R₃; R₂ independently represents a substituted or un-substituted, branched or unbranched alkyl radical, for example a C₁ to C₂₀ alkyl radical, such as a C₁, C₂, C₃, C₄, C₅ or C₆ radical; and R₃ independently represents a group represented by the following Formula II:

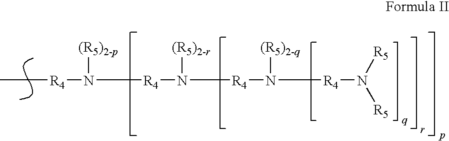

wherein p, q and r independently represent an integer from 0-2, for example, 0, 1 or 2; R₄ independently represents

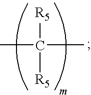

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In another aspect, the invention provides methods of treating an animal, including a human. The method generally involves administering an effective amount of an amine polymer described herein.

Another aspect of the invention is a pharmaceutical composition comprising one or more amine polymers of the present invention with at least one pharmaceutically acceptable carrier. The amine polymers described herein have several therapeutic applications. For example, the amine polymers are useful in removing compounds or ions such as anions, for example phosphorous-containing compounds or phosphorous containing ions such as organophosphates and/or phosphates, from the gastrointestinal tract, such as from the stomach, small intestine and/or large intestine. In some embodiments, the amine polymers are used in the treatment of phosphate imbalance disorders and renal diseases.

In some embodiments, the invention comprises an amine compound or amine polymer that comprises an amine dendrimer or residue thereof, where the dendrimer comprises a polyhydroxy core and branches emanating from the core, where the branches are based on substituted or un-substituted α, β unsaturated nitrile units. The branches may be formed using a reiterative reaction sequence that includes a Michael addition of the substituted or un-substituted α, β unsaturated nitrile and a reduction of the nitrile group to a primary amine.

In yet another aspect, the amine polymers are useful for removing other solutes, such as chloride, bicarbonate, and/or oxalate containing compounds or ions. Amine polymers removing oxalate compounds or ions find use in the treatment of oxalate imbalance disorders. Amine polymers removing chloride compounds or ions find use in treating acidosis, for example. In some embodiments, the amine polymers are useful for removing bile acids and related compounds.

The invention further provides compositions containing any of the above amine polymers where the amine polymer is in the form of particles and where the particles are encased in one or more shells.

In another aspect, the invention provides pharmaceutical compositions. In one embodiment, the pharmaceutical composition contains an amine polymer of the invention and a pharmaceutically acceptable excipient. In some embodiments, the composition is a liquid formulation in which the amine polymer is dispersed in a liquid vehicle, such as water, and suitable excipients. In some embodiments, the invention provides a pharmaceutical composition comprising an amine polymer for binding a target compound or ion, and one or more suitable pharmaceutical excipients, where the composition is in the form of a tablet, sachet, slurry, food formulation, troche, capsule, elixir, suspension, syrup, wafer, chewing gum or lozenge. In some embodiments the composition contains a pharmaceutical excipient selected from the group consisting of sucrose, mannitol, xylitol, maltodextrin, fructose, sorbitol, and combinations thereof. In some embodiments the target anion of the amine polymer is an organophosphate and/or phosphate. In some embodiments the amine polymer is more than about 50% of the weight of the tablet. In some embodiments, the tablet is of cylindrical shape with a diameter of from about 12 mm to about 28 mm and a height of from about 1 mm to about 8 mm and the amine polymer comprises more than 0.6 to about 2.0 gm of the total weight of the tablet.

In some of the compositions of the invention, the excipients are chosen from the group consisting of sweetening agents, binders, lubricants, and disintegrants. Optionally, the amine polymer is present as particles of less than about 80 μm mean diameter. In some of these embodiments, the sweetening agent is selected from the group consisting of sucrose, mannitol, xylitol, maltodextrin, fructose, and sorbitol, and combinations thereof.

In some embodiments, the invention provides amine compounds, amine polymers or compositions that comprise an amine dendrimer or residue thereof, where the amine dendrimer is formed from a core that comprises a sugar alcohol that is substituted with one or more amine groups represented by the following Formula II:

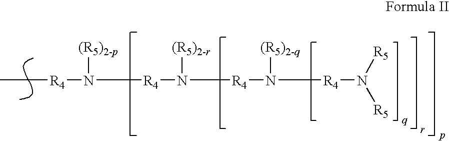

wherein p, q and r independently represent an integer from 0-2, for example, 0, 1 or 2; R₄ independently represents

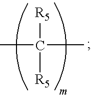

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the invention comprises an amine compound or amine polymer that comprises an amine dendrimer or residue thereof, where the dendrimer comprises a substituted sugar alcohol. The substituted sugar alcohol may be a reaction product of a sugar alcohol and a substituted or un-substituted α, β unsaturated nitrile that is subsequently hydrogenated to form a substituted sugar alcohol having one or more generations of dendritic branching. The dendritic branching may connect to the sugar alcohol core via an ether linkage between one or more hydroxyloxygen atoms of the sugar alcohol and one or more alkylamine groups.

In still other embodiments, a polymer network may include two or more polymers, where at least one of the polymers is an amine polymer derived from an amine compound represented by Formula I, that may be linked to form a polymer network. For example, in some embodiments a polymer network may comprise a residue of two or more sugar alcohols, a residue of one or more substituted or un-substituted α, β unsaturated nitrile groups and a residue of one or more crosslinking agents. In some embodiments, the polymer network may be formed where all or substantially all of the polymers may be amine polymers that are derived from amine compounds represented by Formula I.

### DETAILED DESCRIPTION OF THE INVENTION

In one aspect, the present invention provides amine compounds, amine polymers, compositions and methods of using amine polymers or compositions comprising an amine polymer or amine compound or residue thereof, where the amine compound is represented by Formula I. In some embodiments, the compositions may comprise amine polymers that may be derived from two or more of the amine compounds described herein.

In addition, some embodiments may include multiple amine compounds or residues thereof that repeat in a copolymer or polymer. Such polymers may include one or more additional compounds that may be included in a polymer backbone or as pendant groups either individually or as repeating groups, and that may provide separation between the individual amine polymers.

As used herein, unless otherwise stated, the term “derived from” is understood to mean: produced or obtained from another substance by chemical reaction, especially directly derived from the reactants, for example a substituted sugar alcohol may be derived from the reaction of a sugar alcohol and a substituted or un-substituted α, β unsaturated nitrile that is subsequently hydrogenated to form a substituted sugar alcohol having one or more generations of dendritic branching. Additionally, a substituted sugar alcohol that is reacted with a linking agent, such as a crosslinking agent results in an amine polymer that is derived from the substituted sugar alcohol and the linking agent.

In some embodiments, the invention is an amine compound, amine polymer or composition, or a method for removing a compound or ion, such as a phosphorous-containing compound or a phosphorous-containing ion (e.g. phosphate), from the gastrointestinal tract of an animal by administering an effective amount of an amine polymer that comprises at least one amine compound or residue thereof, where the amine compound is represented by Formula I, as follows:

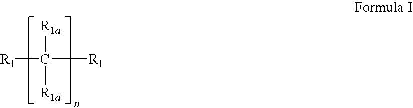

wherein n independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₁, independently represents a hydrogen radical, a hydroxyl radical or —OR₃; R₁ₐ independently represents R₁, —R₂OH or —R₂OR₃; with the proviso that the amine compound includes at least one moiety represented by R₃; R₂ independently represents a substituted or un-substituted, branched or unbranched alkyl radical, for example a C₁ to C₂₀ alkyl radical, such as a C₁, C₂, C₃, C₄, C₅ or C₆ radical; and R₃ independently represents a group represented by the following Formula II:

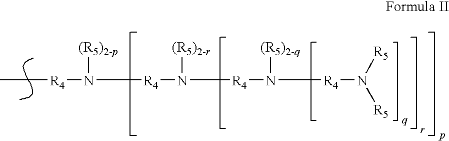

wherein p, q and r independently represent an integer from 0-2, for example, 0, 1 or 2; R₄ independently represents

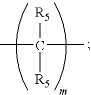

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula I, wherein at least one R₁ₐ comprises R₁.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula I and where R₃ independently represents a group represented by the following Formula IIa:

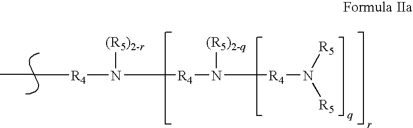

where q, r, R₄ and R₅ are as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula I and where R₃ independently represents a group represented by the following Formula IIb:

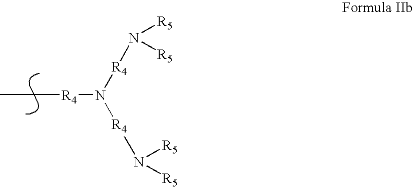

where R₄ and R₅ are as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula III, as follows:

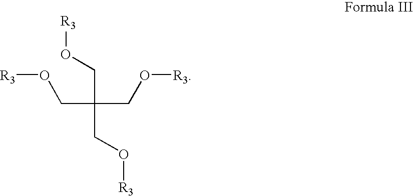

where R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal, such as from the stomach by administering an effective amount of an amine polymer that comprises at least one amine compound or residue thereof, where the amine compound is represented by Formula IV, as follows:

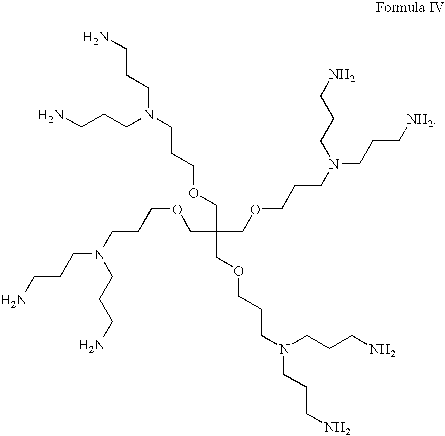

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula V, as follows:

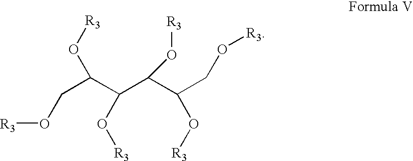

wherein R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula VI, as follows:

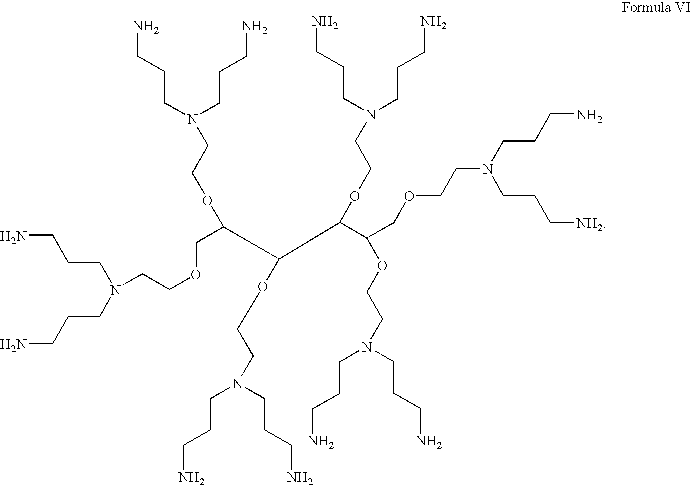

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula VII, as follows:

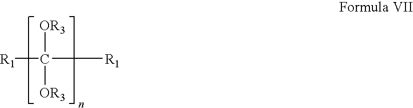

where n and R₁ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula VIII, as follows:

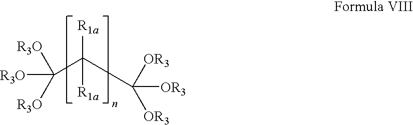

where n and R₁ₐ are as defined above and R₃ independently represents a group represented by Formula II, Formula IIa, or Formula III) as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula IX, as follows:

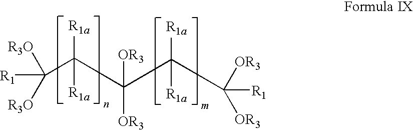

where n, m, R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula X, as follows:

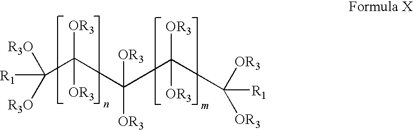

where n, m and R₁ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XI, as follows:

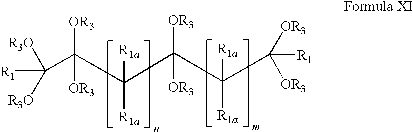

where n, m, R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XII, as follows:

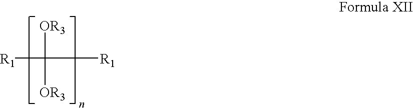

where n and R₁ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XIII, as follows:

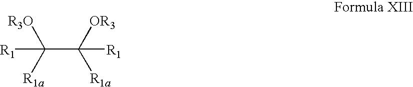

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XIV, as follows:

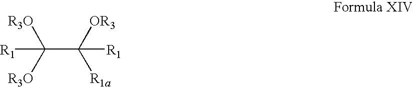

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XV, as follows:

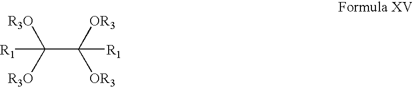

where R₁ is as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XVI, as follows:

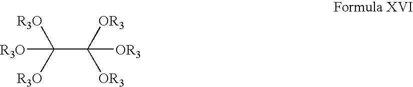

where R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XVII, as follows:

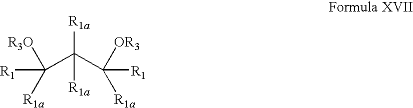

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XVIII, as follows:

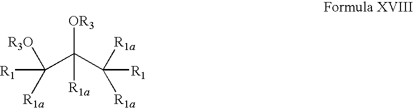

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XIX, as follows:

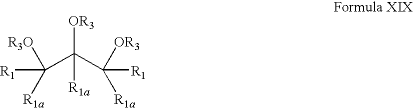

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XX, as follows:

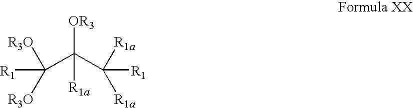

where R₁ and R₁ₐ are as defined above and R₃ independently represents a group represented by Formula II, Formula IIa, or Formula IIb) as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXI, as follows:

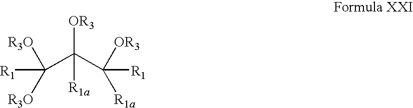

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXII, as follows:

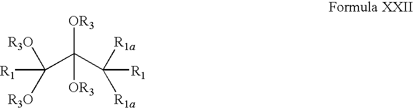

where R₁ and R₁₆ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXIII, as follows:

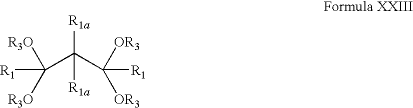

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXIV, as follows:

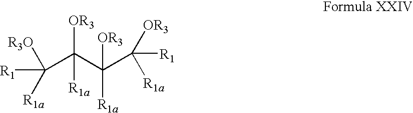

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXV, as follows:

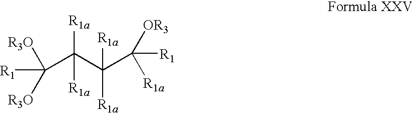

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXVI, as follows:

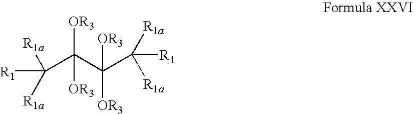

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXVII, as follows:

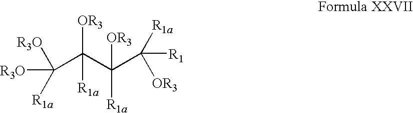

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXVIII, as follows:

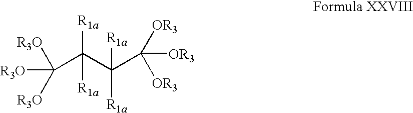

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXIX, as follows:

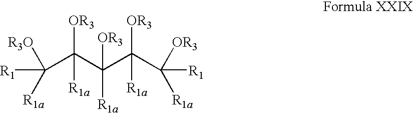

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXX, as follows:

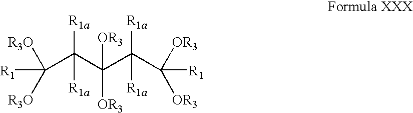

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXI, as follows:

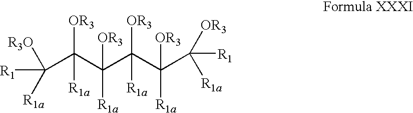

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXII, as follows:

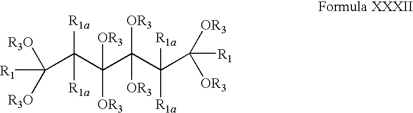

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXIII, as follows:

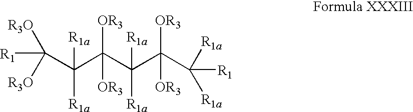

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXIV, as follows:

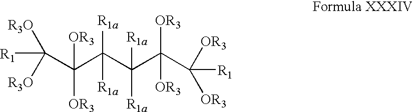

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXV, as follows:

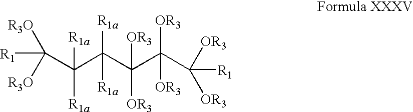

where R₁, and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXVI, as follows:

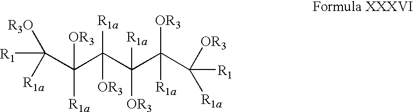

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXVII, as follows:

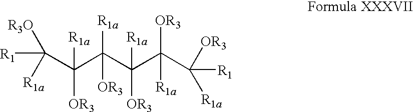

where R₁ and R₁ₐ are as defined above and R₃ independently represents a group represented by Formula II, Formula IIa, or Formula IIb as defined above.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound is represented by Formula XXXVIII, as follows:

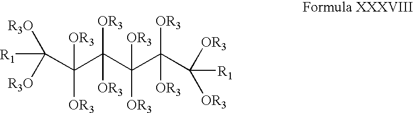

where R₁ and R₃ are as defined above for Formula I.

Some embodiments of the invention include a pharmaceutical composition that comprises an amine compound or residue thereof or an amine polymer or residue thereof, where the amine polymer comprises an amine compound or residue thereof, the amine compound comprising one or more sugar alcohols substituted with an amine group represented by the following Formula II:

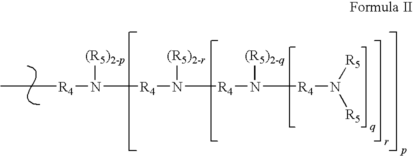

wherein p, q and r independently represent an integer from 0-2, for example, 0, 1 or 2; R₄ independently represents

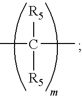

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound comprises a substituted sugar alcohol having one or more units represented by the group comprising the following Formula XXXIX:

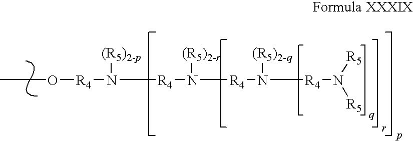

wherein p, q and r independently represent an integer from 0-2, for example, 0, 1 or 2; R₄ independently represents

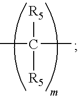

wherein m independently represents an integer from 1-20, for example, 1-15, 1-2, 3-6, 7-10, 11-15, such as 1, 2, 3, 4, 5, 6, 7, 8, 9, 10, 11, 12, 13, 14, 15, 16, 17, 18, 19 or 20; R₅ independently represents a hydrogen radical; a substituted or un-substituted alkyl radical; a substituted or un-substituted aryl radical; or R₅ and a neighboring R₅ together represent a link or links comprising a residue of a crosslinking agent, for example epichlorohydrin or other crosslinking agents, a substituted or un-substituted alicyclic radical, a substituted or un-substituted aromatic radical, or a substituted or un-substituted heterocyclic radical; or R₅ represents a link with another compound or a residue thereof.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal by administering an effective amount of an amine polymer that comprises at least one amine compound or residue thereof, where the amine compound comprises an amine dendrimer or residue thereof, the dendrimer having a core that is a residue of one or more sugar alcohols and a residue of one or more substituted or un-substituted α, β unsaturated nitriles.

In some embodiments, dendrimers of the present invention may be formed from any suitable reaction scheme. Dendrimers are macromolecular compounds that comprise a core that includes functional groups and dendritic branches that may be formed through a series of iterative reaction sequences with the functional groups on the core to form a branched macromolecule. In some embodiments the reactive functional groups comprise hydroxyl groups and/or amine groups. The functional groups will have functionalities that are dependent on the type of group. For example, hydroxyl groups have a functionality of one, while primary amines generally have a functionality of 2, though they may be quaternized. In some embodiments, an amine polymer comprises a dendrimer or residue thereof where the dendrimer comprises a polyhydroxy core that comprises a residue of one or more hydroxyl groups and a residue of one or more substituted or un-substituted α, β unsaturated nitrite groups, the amine polymer further comprising a crosslinking or other linking agent or residue thereof. Some examples of substituted or un-substituted α, β unsaturated nitriles include methacrylonitrile and acrylonitrile.

In some embodiments, dendrimers of the present invention are prepared by a Michael addition of a substituted or un-substituted α, β unsaturated nitrite to one or more of the hydroxyl groups on a polyhydroxy core to replace the hydrogen of the hydroxyl group with an nitrile group resulting in an ether linkage to the core via the oxygen atom of the hydroxyl group. The nitriles of the nitrile groups of the resulting compound are then chemically reduced, for example via hydrogenation, to form the corresponding primary amines. The Michael addition and subsequent reduction may be repeated on the primary amines generally yielding a branched tertiary amine. Subsequent Michael additions and reductions may be repeated one or more times to provide the branched structure characteristic of dendrimers. A schematic of this process is provided below in Scheme I, using acrylonitrile as the substituted or un-substituted α, β unsaturated nitrile and mannitol as the polyol:

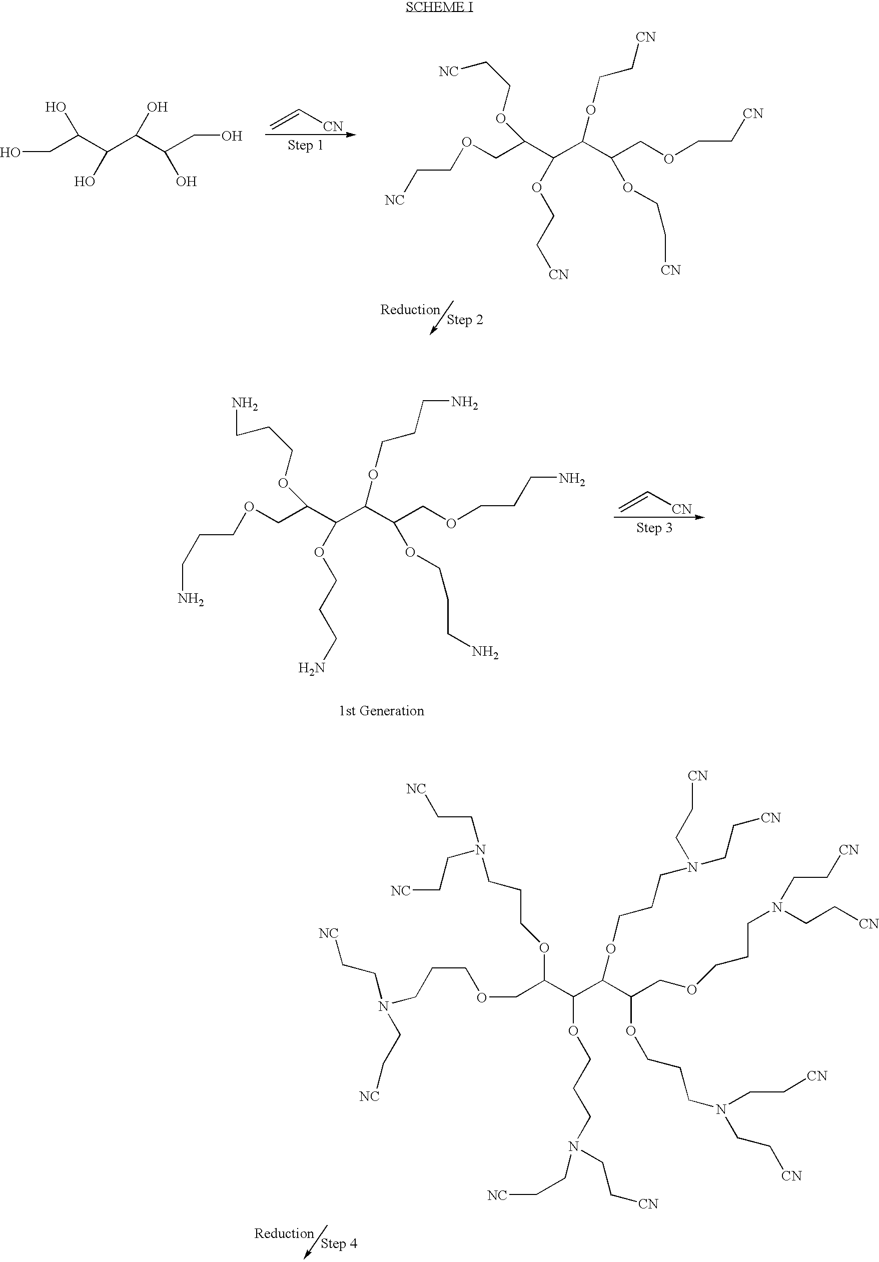

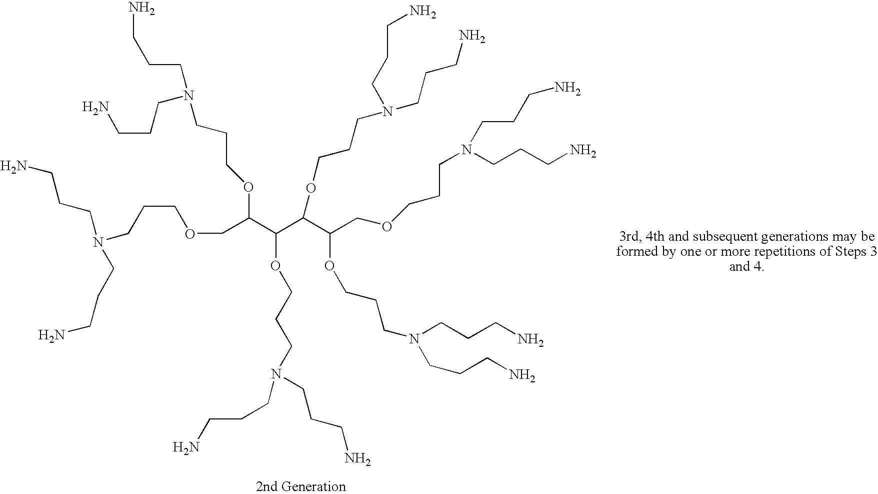

In some embodiments, each iteration of Michael addition and subsequent reduction may be considered one generation. Thus, for some embodiments, a compound having one generation of dendritic branching may have undergone one iteration of Michael addition and reduction, compounds having two generations of dendritic branching may have undergone two iterations of Michael addition and reduction, compounds having three generations of dendritic branching may have undergone three iterations of Michael addition and reduction, compounds having four generations of dendritic branching may have undergone four iterations of Michael addition and reduction, etc. Generally dendrimers according to some embodiments of the present invention may have from 1-10, such as 2, 3, 4, 5, 6, 7, 8, or 9 generations of dendritic branching.

In some embodiments, a method of making an amine polymer comprises reacting a polyhydroxy core with a substituted or un-substituted α, β unsaturated nitrile using a Michael addition reaction to form a polyether, reducing at least one nitrile group on the polyether to form a tertiary amine, repeating the Michael addition and reduction on the tertiary amine one or more times to form an amine dendrimer; and crosslinking the amine dendrimer with a crosslinking agent.

Some embodiments of the invention may comprise a polymer network or composition or a method for removing a compound or ion, such as a phosphorous-containing compound or a phosphorous-containing ion (e.g. phosphate) from the gastrointestinal tract of an animal by administering an effective amount of a polymer network that comprises two or more amine compounds, amine polymers or residues thereof, that comprise a residue of two or more substituted or un-substituted sugar alcohols, a residue of one or more substituted or un-substituted α, β unsaturated nitrile groups and a residue of one or more crosslinking or other linking agents. In some embodiments, the polymer network comprises residues of two or more polyethers, where the polyethers comprise a residue of one or more substituted or un-substituted sugar alcohols and a residue of one or more substituted or un-substituted α, β unsaturated nitrite groups, and where the network also comprises a residue of one or more crosslinking agents. In some embodiments, the polymer network may include one or more amine dendrimers or residues thereof.

In some embodiments, the invention is an amine compound, amine polymer or composition, or a method for removing a compound or ion, such as a phosphorous-containing compound or a phosphorous-containing ion (e.g. phosphate) from the gastrointestinal tract of an animal, such as from the stomach by administering an effective amount of two or more amine dendrimers or residues thereof represented by Formula V:

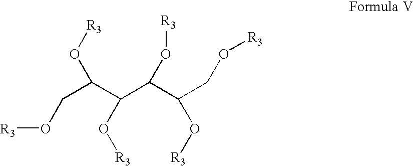

wherein R₃ independently represents a group represented by Formula II, Formula IIa, or Formula IIb as defined above.

In some embodiments, the invention is an amine compound, amine polymer, composition, polymer network or a method for removing a compound or ion, such as a phosphorous-containing compound or a phosphorous-containing ion (e.g. phosphate) from the gastrointestinal tract of an animal, by administering an effective amount of an amine polymer, polymer network or composition having a plurality of units represented by the following Formula XL:

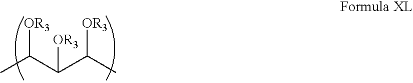

wherein R₃ independently represents a group represented by Formula II, Formula IIa, or Formula IIb as defined above; and a plurality of units represented by the following Formula XLI:

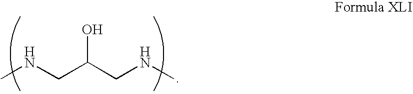

In some embodiments, the invention is a method for reducing blood phosphate levels by 5-100% in a patient in need thereof, the method comprising administering a therapeutically effective amount of an amine polymer or composition to the patient, where the amine polymer or composition comprises an amine compound according to Formula I or a residue thereof. In some embodiments, the invention is a method for reducing urinary phosphorous by 5-100% in a patient in need thereof, the method comprising administering a therapeutically effective amount of an amine polymer or composition to the patient, where the amine polymer or composition comprises an amine compound according to Formula I or a residue thereof.

In some embodiments, the invention is a method of treating a phosphate imbalance disorder such as hyperphosphatemia comprising administering a therapeutically effective amount of an amine polymer or composition to a patient in need thereof. In some embodiments, the amine polymer or composition comprises an amine compound or residue thereof according to Formula I. In some embodiments, a method of treating a phosphate imbalance disorder such as hyperphosphatemia comprises administering a therapeutically effective amount of an amine polymer or composition to a patient in need thereof, where the amine polymer or composition comprises an amine compound or residue thereof represented by any of Formulas III-XXXVIII, or where the amine polymer or composition comprises plurality of units according to Formula XXXIX, or a plurality of units according to Formulas XL and XLI.

In some embodiments, the amine compound is a mixture of more than one amine compound, for example 2-20 such as 2, 3, 4, 5, 6, 7, 8, 9 or 10 amine compounds, represented by Formulas I and/or III-XXXVIII. In some embodiments, the mixture predominantly comprises an amine compound represented by one of Formulas I, III, V or VII-XXXVIII where q, r and p are independently 0 or 2. For example, in some embodiments a plurality of the mixture, such as greater than 30 wt. %, greater than 40 wt. %, greater than 50 wt. %, greater than 60 wt. % or greater than 70 wt. % based on the total weight of the mixture, comprises an amine compound or residue thereof represented by one of Formulas I, III, V or VII-XXXVIII where q, r and p are independently 0 or 2. For example, in some embodiments, the mixture comprises greater than 30 wt %, greater than 40 wt. %, greater than 50 wt. %, greater than 60 wt. % or greater than 70 wt. % of an amide compound or residue thereof represented by Formula IV or Formula VI.

In some embodiments, the invention comprises an amine polymer derived from an amine compound that is a mixture of amine compounds, a pharmaceutical composition comprising such an amine polymer, or a method of using the same in a therapeutically effective amount to remove a compound or ion, such as a phosphorous-containing compound or a phosphorous-containing ion (e.g. phosphate), from the gastrointestinal tract of an animal.

Polyhydroxy compounds that may be used as cores for, or in the preparation of amine compounds, amine polymers, polymer networks and compositions according to some embodiments of the invention include straight chain, branched, cyclic, alicyclic, aromatic, and heterocyclic polyhydric alcohols, such as 1,4-butanediol, 1,5-pentanediol, 1,6-hexanediol, 1,6-cyclohexanedimethanol, 2-methyl-1,3-propanediol, 2-methyl-2-ethyl-1,3-propanediol, 2-ethyl-2-butyl-1,3-propanediol, neopentyl glycol, dimethylolpropane, 1,1-dimethylolcyclohexane, glycerol, trimethylolethane, trimethylolpropane, diglycerol, ditrimethylolethane, ditrimethylolpropane, pentaerythritol, dipentaerythritol, inositol.

Examples of some aromatic, alicyclic and heterocyclic groups that may be substituted with at least 2 hydroxyl groups to form suitable polyhydroxy compounds include cyclopropyl, cyclobutyl, cyclopentyl, cyclohexyl, piperidinyl, piperizinyl, thiazolidinyl, imidazolidinyl, pyranyl, tetrahydrofuranyl, oxanyl, benzyl, pyridinyl, imidazolyl, pyrazolyl, thiazolyl, oxazolyl, pyrimidinyl, dioxanyl, quinizolinyl, indolinyl, benzothiazolyl, benzooxazolyl, pyrazinyl, furanyl, thenyl, naphthalenyl and the like.

Non-limiting examples of some suitable cyclic polyhydroxy compounds include: cyclohexane-1,2-diol, cyclohexane-1,3-diol, cyclohexane-1,4-diol, cyclohexane-1,2,3-triol, cyclohexane-1,2,4-triol, cyclohexane-1,3,4-triol, cyclohexane-1,3,5-triol, cyclohexane-1,2,3,4-tetraol, cyclohexane-1,3,4,5-tetraol, cyclohexane-1,2,3,4,5-pentaol, cyclohexane-1,2,3,4,5,6-hexaol, cyclopentane-1,2-diol, cyclopentane-1,3-diol, cyclopentane-1,2-diol, cyclopentane-1,2,3-triol, cyclopentane-1,2,4-triol, cyclopentane-1,2,3,4-tetraol, cyclopentane-1,2,3,4,5-pentaol, benzene-1,2-diol, benzene-1,3-diol, benzene-1,4-diol, benzene-1,2,3-triol, benzene-1,2,4-triol, benzene-1,3,4-triol, benzene-1,3,5-triol, benzene-1,2,3,4-tetraol, benzene-1,3,5-triol, benzene-1,2,3,4-tetraol, benzene-1,2,3,5-tetraol, benzene-1,2,4,5-tetraol, benzene-1,2,3,4,5-pentaol, benzene-1,2,3,4,5,6-hexaol, and sugar alcohols

Sugar alcohols that are suitable for use alone or in combination in some embodiments of the amine compounds, amine polymers or compositions of the present invention include monosaccharides and sugar alcohols derived from monosaccharides. Examples of such compounds include sugar alcohols comprising or derived from aldoses and ketoses including those comprising or derived from monoses, dioses, trioses, tetroses, pentoses, hexoses, heptoses, octoses and nonoses. The aldoses and ketoses which the sugar alcohols comprise or from which the sugar alcohols are derived may be fully or partially hydrogenated, and may be substituted, including replacement of one or more hydroxyl groups on the aldose or ketose with one or more hydrogen groups to form the corresponding deoxyaldose or deoxyketose, provided that at least one alcohol group remains and substitution of one or more hydroxyl groups with one or more amine groups to form the corresponding amino sugar. Specific non-limiting examples of some aldoses and ketoses include: erythrose, threose, ribose, deoxyribose, arabinose, xylose, lyxose, allose, altrose, glucose, mannose, gulose, idose, galactose, talose, ribulose, rhamnose, fucose, ribodesose, xylulose, fructose, psicose, tagatose, mannoheptulose, sedoheptulose, sorbose, pentaerythrose, octolose, sialose, glucosamine, glucosylamine, mannosamine, galactosamine, allosamine, altrosamine, ribosamine, arabinosamine, gulosamine, idosamine, talosamine, xylosamine, lyxosamine, sorbosamine, tagatosamine, psicosamine, fructosamine, and sialic acids, including both the D and L forms of each, α and β forms of each, partially or fully hydrogenated derivatives thereof, or combinations thereof. Non-limiting examples of some suitable sugar alcohols include sorbitol, mannitol, xylitol, erythritol, galactitol, dulcitol, arabitol, threitol, arabinitol, ribitol, and rhamnitol.

In some embodiments, suitable polyhydroxy compounds include one or more substituted or unsubstituted cyclic sugars or cyclic sugar alcohols such as cyclic forms of aldoses and ketoses, including cyclic forms of the aldoses and ketoses described above. Other suitable cyclic polyols that may be used alone or in combination include substituted or unsubstituted polysaccharides, including disaccharides and oligosaccharides, including hetero and homopolysaccharides derived from cyclic forms of the aldoses and ketoses described herein. Such polysaccharides may be unbranched or branched and may include α and/or β glycosidic bonds such as, for example, α(1→4), α(1→0), α(1→6), α(1→3), β(1→3) and/or β(1 →4) glycosidic bonds. In unsubstituted form, polysaccharides may have the general formula Cₙ(H₂O)ₙ₋₁, where n is from 6-3000. Non-limiting examples of some substituted or unsubstituted polysaccharides include: sucrose, maltose, chitobiose, laminarbiose, kojibiose, xylobiose, trehalose, saccharose, cellobiose, gentiobiose, lactose, melibiose, raffinose, gentianose, melizitose, stachyose, inulin, methyl-α-glucopyranoside, amylosamine, maltosamine, agarosamine, cellulosamine, saccharosamine, starches, amylose, amylopectin, pectins/pectic polysaccharides, arabingalactans, mannans, mucopolysaccharides, hyaluronic acid, heparin, glucomannans, celluloses, chitins, glycogen, callose, laminarin, xylan and glactomannan.

Examples of some suitable polyhydroxy compounds include the following compounds:

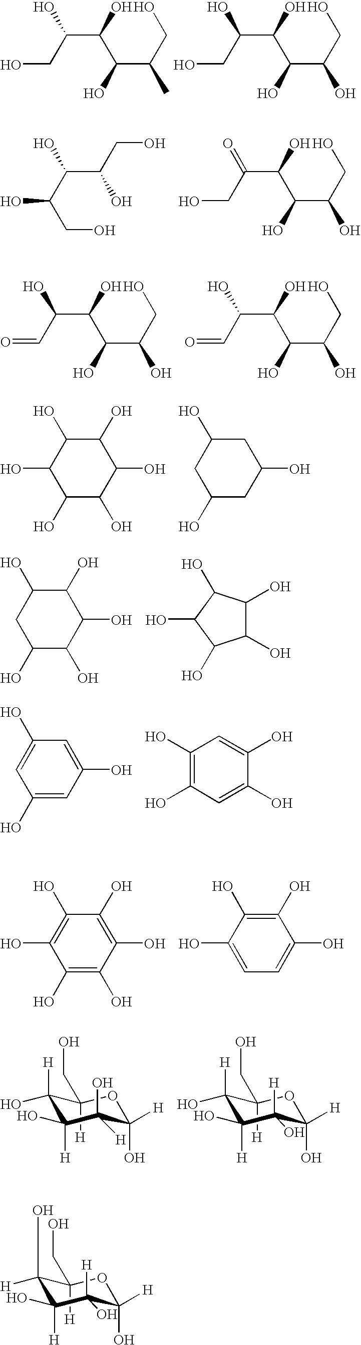

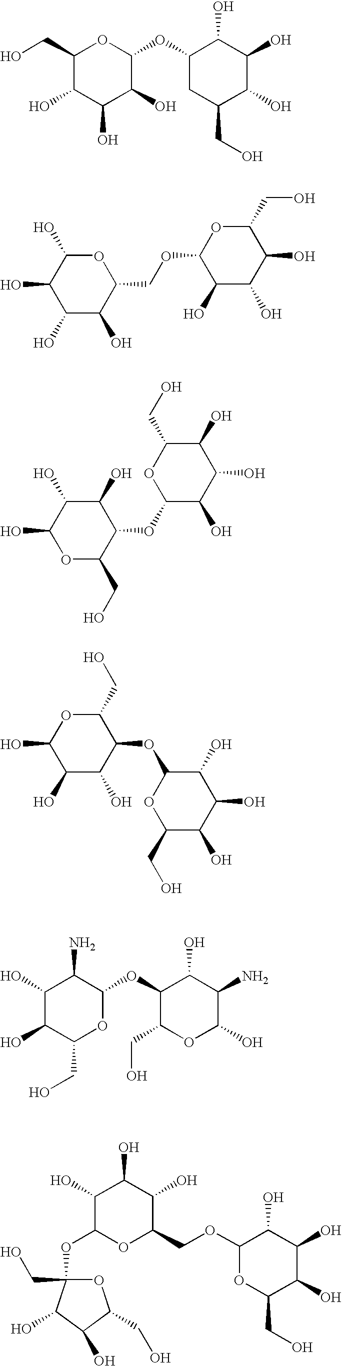

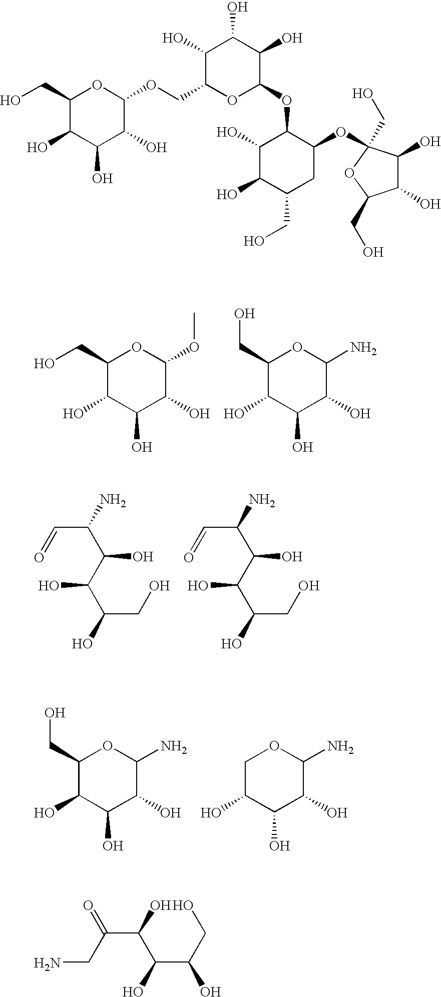

Other embodiments of the invention include pendant amine polymers formed with amine compounds or residues thereof as pendant groups on a polymer or polymerized backbone of a polymer. Such pendant amine polymers may be formed by adding one or more polymerizable groups to one or more amine groups on an amine compound to form an amine monomer and then subsequently polymerizing the polymerizable group to form a pendant amine polymer comprising an amine compound or residue thereof. A schematic example of such an addition follows [it should be noted in the following that an amine compound designated as “AC” is intended to represent an amine compound or residue thereof, of the invention, with one of its amine groups depicted for purposes of illustrating how a polymerizable group may be added to an amine compound]:

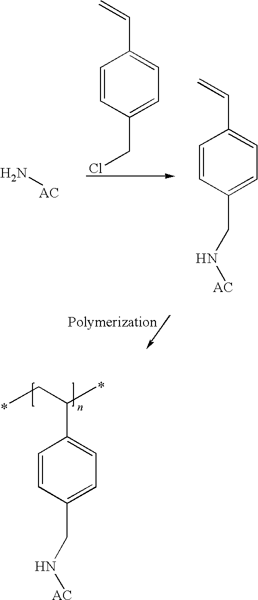

Non-limiting examples of other polymerizable groups that may be used with amine compounds or residues thereof according to embodiments of the invention include:

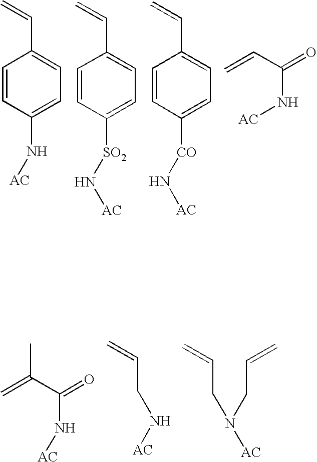

One or more polymerizable groups may be added to each amine compound and thus it is possible to have mixtures of amine monomers having various pendant ACs having differing numbers of polymerizable groups. In addition, the pendant amine polymers made in this fashion may be modified, crosslinked, formed into a network or substituted post polymerization. Such modification may be performed for any number of reasons, including to improve efficacy, tolerability or reduce side effects.

Amine monomers may also be formed by addition of amine compounds to amine-reactive polymers by reacting one or more amine groups of the amine monomers with one or amine-reactive groups on the amine-reactive polymers. Examples of some amine reactive polymers include:

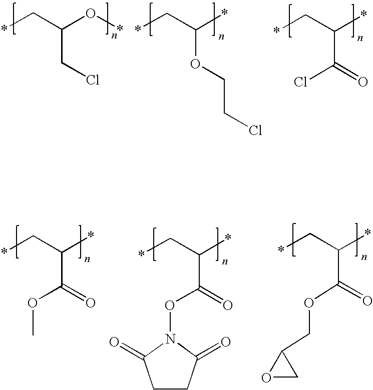

The amine compounds or amine monomers may also serve as multifunctional amine monomers to form polymers. For example, when the amine compounds or the polymers formed from the amine monomers are crosslinked, the crosslinking reaction may be carried out either in solution of bulk (i.e. using the neat amine and neat crosslinking agents) or in dispersed media. When a bulk process is used, solvents are selected so that they co-dissolve the reactants and do not interfere with the crosslinking reaction. Suitable solvents include water, low boiling alcohols (methanol, ethanol, butanol), dimethylformamide, dimethylsulfoxide, acetone, methylethylketone, and the like.

Other polymerization methods may include a single polymerization reaction, stepwise addition of individual monomers via a series of reactions, the stepwise addition of blocks of monomers, combinations of the foregoing, or any other method of polymerization, such as, for example, direct or inverse suspension, condensation, emulsion, precipitation techniques, polymerization in aerosol or using bulk polymerization/crosslinking methods and size reduction processes such as extrusion and grinding. Processes can be carried out as batch, semi-continuous and continuous processes. For processes in dispersed media, the continuous phase can be selected from apolar solvents such as toluene, benzene, hydrocarbon, halogenated solvents, supercritical carbon dioxide, and the like. With a direct suspension process, water can be used, although salt brines are also useful to “salt out” the amine and crosslinking agents in a droplet separate phase.

Amine compounds and amine monomers of the invention may be copolymerized with one or more other monomers or oligomers or other polymerizable groups, may be crosslinked, may have crosslinking or other linking agents or monomers within the polymer backbone or as pendant groups or may be formed or polymerized to form a polymer network or mixed polymer network comprising: amine compounds or residues thereof, amine monomers or residues thereof, crosslinking agent or residues thereof, or other linking agents or residues thereof. The network may include multiple connections between the same or different molecules that may be direct or may include one or more linking groups such as crosslinking agents or other linking agents such as monomers or oligomers or residues thereof.

Non-limiting examples of comonomers which may be used alone or in combination include: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyle, methallyl monomers and combinations thereof. Functionalized versions of these monomers may also be used. Additional specific monomers or comonomers that may be used in this invention include, but are not limited to, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N—N-butylmethacrylamide, N-methylolmethacrylamide, N-ethylolmethacrylamide, N-tert-butylacrylamide, N—N-butylacrylamide, N-methylolacrylamide, N-ethylolacrylamide, 4-acryloylmorpholine, vinyl benzoic acid (all isomers), diethylaminostyrene (all isomers), α-methylvinyl benzoic acid (all isomers), diethylamino α-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate and combinations thereof.

In some embodiments, amine polymers of the invention are crosslinked using crosslinking agents, and may not dissolve in solvents, and, at most, swell in solvents. The swelling ratio may be measured according to the procedure in the Test Methods section below and is typically in the range of about 1 to about 20; for example 2 to 10, 2.5 to 8, 3 to 6 such as less than 5, less than 6, or less than 7. In some embodiments, the amine polymers may include crosslinking or other linking agents that may result in amine polymers that do not form gels in solvents and may be soluble or partially soluble in some solvents.

Crosslinking agents are typically compounds having at least two functional groups that are selected from a halogen group, carbonyl group, epoxy group, ester group, acid anhydride group, acid halide group, isocyanate group, vinyl group, and chloroformate group. The crosslinking agent may be attached to the carbon backbone or to a nitrogen of an amine compound, amine monomer or residue thereof.

Examples of crosslinking agents that are suitable for synthesis of the polymers or dendrimers of the present invention include, but are not limited to, one or more multifunctional crosslinking agents such as: dihaloalkanes, haloalkyloxiranes, alkyloxirane sulfonates, di(haloalkyl)amines, tri(haloalkyl)amines, diepoxides, triepoxides, tetraepoxides, bis(halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy)diphenylmethane, bisphenol F diglycidyl ether, 1,4-bis(2′,3′-epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4h-chromene-2-carboxylate, bis[4-(2,3-epoxy-propylthio)phenyl]-sulfide, 1,3-bis(3-glycidoxypropyl)tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo[7.3.3.15,11]heptasiloxane, 4,4′-methylenebis(N,N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate. When the crosslinking agent is an alkylhalide compound, a base can be used to scavenge the acid formed during the reaction. Inorganic or organic bases are suitable. NaOH is preferred. The base to crosslinking agent ratio is preferably between about 0.5 to about 2.

In some embodiments, the crosslinking agents may be introduced into the polymerization reaction in an amount of from 0.5 to 25 wt. % based on the total weight of the amine polymer or polymer, such as from about 2 to about 15 wt. %, from about 2 to about 12 wt. %, from about 3 to about 10 wt. %, or from about 3 to about 6 wt. %, such as 2, 3, 4, 5, 6 wt %. The amount of crosslinking agent necessary may depend on the extent of branching within the amine compound.

In some embodiment the molecular weight of the amine polymers, may be typically at least about 1000. For example, the molecular weight may be from about 1000 to about 1,000,000, such as about 1000 to about 750,000, about 1000 to about 500,000, about 1000 to about 250,000, about 1000 to about 100,000 such as less than 750,000, less than 500,000, 250,000 or less than 100,000.

In some embodiments, the pharmaceutical composition of the present invention comprises an amine polymer comprising at least one amine compound or residue thereof, where the amine compound is represented by Formula III where R₅ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising at least one amine compound or residue thereof, where the amine compound is represented by Formula III where R₅ independently represents a H radical or alkyl radical, q is 0 and r and p both are 2, m independently represents an integer from 3-6, such as 3, 4, 5 or 6, where the compound is crosslinked with a crosslinking agent as defined above in this paragraph. A further pharmaceutical composition embodiment of the present invention comprises an amine polymer comprising at least one amine compound or residue thereof, where the amine compound is represented by Formula III where R₅ independently represents a H radical or alkyl radical, q, r and p are each 2, m independently represents an integer from 3-6, such as 3, 4, 5 or 6, where the compound is crosslinked with a crosslinking agent as defined above in this paragraph.

In some embodiments, the pharmaceutical composition of the present invention comprises an amine polymer comprising at least one amine compound or residue thereof, where the amine compound is represented by Formula V where R₅ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising at least one amine compound or residue thereof, where the amine compound is represented by Formula V, where R₅ independently represents a H radical or alkyl radical, q is 0 and r and p both are 2, m independently represents an integer from 3-6, such as 3, 4, 5 or 6, where the compound is crosslinked with a crosslinking agent as defined above in this paragraph. A further pharmaceutical composition embodiment of the present invention comprises an amine polymer comprising at least one amine compound or residue thereof, where the amine compound is represented by Formula V where R₅ independently represents a H radical or alkyl radical, q, r and p are each 2, m independently represents an integer from 3-6, such as 3, 4, 5 or 6, where the compound is crosslinked with a crosslinking agent as defined above in this paragraph.

Another pharmaceutical composition of the present invention comprises an amine polymer comprising an amine compound or residue thereof, the amine compound comprising a substituted sugar alcohol having one or more units represented by Formula XXXIX where R₅ independently represents a H radical or alkyl radical, q and r are 0 and p is 2, m independently represents an integer from 3-6, such as 3, 4, 5 or 6; and 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer comprising an amine compound or residue thereof, the amine compound comprising substituted sugar alcohol having one or more units represented by Formula XXXIX where R₅ independently represents a H radical or alkyl radical, q is 0 and r and p both are 2, m independently represents an integer from 3-6, such as 3, 4, 5 or 6, and crosslinked with a crosslinking agent as defined above in this paragraph. A further pharmaceutical composition embodiment of the present invention comprises an amine polymer comprising an amine compound or residue thereof, the amine compound comprising substituted sugar alcohol having one or more units represented by Formula XXXIX where R₅ independently represents a H radical or alkyl radical, q, r and p are each 2, m independently represents an integer from 3-6, such as 3, 4, 5 or 6, and crosslinked with a crosslinking agent as defined above in this paragraph.

In some embodiments, the invention is a compound or composition or method for removing an anion, such as organophosphate or phosphate, from the gastrointestinal tract of an animal by administering an effective amount of an amine polymer that comprises an amine dendrimer having a core that is a residue of one or more sugar alcohols and a residue of one or more substituted or un-substituted α, β unsaturated nitriles.

Another pharmaceutical composition of the present invention comprises an amine polymer that comprises an amine dendrimer or residue thereof having a core that is a residue of mannitol, sorbitol or other 6-carbon sugar alcohol and a residue of one or more acrylonitriles; where the dendrimer is crosslinked with 2-6 wt. % crosslinking agent or residue thereof, such as 2 wt. %, 3 wt. %, 4 wt. %, 5 wt. % or 6 wt. % crosslinking agent, where the crosslinking agent is epichlorohydrin, poly(epichlorohydrin), 1,2-dibromoethane, tris(2-chloroethyl)amine or 1,4-butanediol diglycidyl ether. Another pharmaceutical composition embodiment of the present invention comprises an amine polymer that comprises an amine dendrimer or residue thereof having a core that is a residue of pentaerythritol and a residue of one or more acrylonitriles; and where the dendrimer is crosslinked with a crosslinking agent as defined above in this paragraph.

Another pharmaceutical composition of the present invention comprises a polymer network having a plurality of units represented by Formula XL where n is from 3-6, the composition also having a plurality of units represented by Formula XLI.

The polymers of some embodiments may be formed using a polymerization initiator. Generally, any initiator may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′-azobis(isobutyronitrile), 2,2′-azobis(N,N′-dimethyleneisobutyramidine)dihydrochloride, 2,2′-azobis(2-amidinopropane)dihydrochloride, 2,2′-azobis(N,N′-dimethyleneisobutyramidine), 1,1′-azobis(1-cyclohexanecarbo-nitrile), 4,4′-azobis(4-cyanopentanoic acid), 2,2′-azobis(isobutyramide) dihydrate, 2,2′-azobis(2-methylpropane), 2,2′-azobis(2-methylbutyronitrile), VAZO 67, cyanopentanoic acid, the peroxy pivalates, dodecylbenzene peroxide, benzoyl peroxide, di-t-butyl hydroperoxide, t-butyl peracetate, acetyl peroxide, dicumyl peroxide, cumyl hydroperoxide, dimethyl bis(butylperoxy) hexane.

In some embodiments, any of the nitrogen atoms within the amine compounds or residues thereof according to embodiments of the invention may optionally be quaternized to yield the corresponding positively charged tertiary nitrogen group, such as for example, an ammonium or substituted ammonium group. Any one or more of the nitrogen atoms in the amine compound or residue thereof may be quaternized and such quaternization, when present, is not limited to or required to include terminal amine nitrogen atoms. In some embodiments, this quaternization may result in additional network formation and may be the result of addition of crosslinking, linking or amine reactive groups to the nitrogen. The ammonium groups may be associated with a pharmaceutically acceptable counterion.

In some embodiments, amine compounds and amine polymers of the invention may be partially or fully quaternized, including protonated, with a pharmaceutically acceptable counterion, which may be organic ions, inorganic ions, or a combination thereof. Examples of some suitable inorganic ions include halides (e.g., chloride, bromide or iodide) carbonates, bicarbonates, sulfates, bisulfates, hydroxides, nitrates, persulfates and sulfites. Examples of some suitable organic ions include acetates, ascorbates, benzoates, citrates, dihydrogen citrates, hydrogen citrates, oxalates, succinates, tartrates, taurocholates, glycocholates, and cholates. Preferred ions include chlorides and carbonates.

In some embodiments, amine compounds and amine polymers of the invention may be protonated such that the fraction of protonated nitrogen atoms is from 1 to 25%, preferably 3 to 25%, more preferably 5 to 15%.

In one embodiment, a pharmaceutically acceptable amine polymer is an amine polymer in protonated form and comprises a carbonate anion. In one embodiment the pharmaceutically acceptable amine polymer is in protonated form and comprises a mixture of carbonate and bicarbonate anions.

In some embodiments, compounds of the invention are characterized by their ability to bind compounds or ions. Preferably the compounds of the invention bind anions, more preferably they bind organophosphates, phosphate and/or oxalate, and most preferably they bind organophosphates or phosphate. For illustration, anion-binding amine polymers and especially organophosphate or phosphate-binding amine polymers will be described; however, it is understood that this description applies equally, with appropriate modifications that will be apparent to those of skill in the art, to other ions, compounds and solutes. Amine polymers may bind an ion, e.g., an anion when they associate with the ion, generally though not necessarily in a noncovalent manner, with sufficient association strength that at least a portion of the ion remains bound under the in vitro or in vivo conditions in which the polymer is used for sufficient time to effect a removal of the ion from solution or from the body. A target ion may be an ion to which the amine polymer binds, and usually refers to the ion whose binding to the amine polymer is thought to produce the therapeutic effect of the compound and may be an anion or a cation. A compound of the invention may have more than one target ion.

For example, some of the amine polymers described herein exhibit organophosphate or phosphate binding properties. Phosphate binding capacity is a measure of the amount of phosphate ion a phosphate binder can bind in a given solution. For example, binding capacities of phosphate binders can be measured in vitro, e.g., in water or in saline solution, or in vivo, e.g., from phosphate urinary excretion, or ex vivo, for example using aspirate liquids, e.g., chyme obtained from lab animals, patients or volunteers. Measurements can be made in a solution containing only phosphate ion, or at least no other competing solutes that compete with phosphate ions for binding to the amine polymer. In these cases, a non interfering buffer may be used. Alternatively, measurements can be made in the presence of other competing solutes, e.g., other ions or metabolites, that compete with phosphate ions (the target solute) for binding to the amine polymer.

Ion binding capacity for an amine polymer may be measured as indicated in the Test Methods. Some embodiments have a phosphate binding capacity which can be greater than about 0.2, 0.5, 1.0, 1.5, 2.0, 2.5, 3.0, 3.5, 4.0, 5.0, 6.0, 8.0, 10.0, 12, 14, 16, 18 or greater than about 20 mmol/g. In some embodiments, the in vitro phosphate binding capacity of amine polymers of the invention for a target ion is greater than about 0.5 mmol/g, preferably greater than about 2.5 mmol/g, even more preferably greater than about 3 mmol/g, even more preferably greater than about 4 mmol/g, and yet even more preferably greater than about 6 mmol/g. In some embodiments, the phosphate binding capacity can range from about 0.2 mmol/g to about 20 mmol/g, such as about 0.5 mmol/g to about 10 mmol/g, preferably from about 2.5 mmol/g to about 8 mmol/g, and even more preferably from about 3 mmol/g to about 6 mmol/g. Phosphate binding may be measured according to the techniques described in the Test Methods section below.

In some embodiments, amine compounds, polymers and compositions of the invention may reduce urinary phosphorous of a patient in need thereof by 5-100%, such as 10-75%, 25-65%, or 45-60%. Some embodiments may reduce urinary phosphorous by greater than 10%, greater than 20%, greater than 30%, greater than 40%, greater than 45%, greater than 50% or greater than 60%. Reduction of urinary phosphorous may be measured according to the methods detailed in the Test Methods section below.

In some embodiments, amine polymers and compositions of the invention may reduce blood phosphate of a patient in need thereof by 5-100%, such as 10-75%, 25-65%, or 45-60%. Some embodiments may reduce blood phosphate levels by greater than 10%, greater than 20%, greater than 30%, greater than 40%, greater than 45%, greater than 50% or greater than 60%.

When crosslinked, some embodiments of the amine compounds of the invention form a gel in a solvent, such as in a simulated gastrointestinal medium or a physiologically acceptable medium.

One aspect of the invention is core-shell compositions comprising a polymeric core and shell. In some embodiments, the polymeric core comprises the amine polymers described herein. The shell material can be chemically anchored to the core material or physically coated. In the former case, the shell can be grown on the core component through chemical means, for example by: chemical grafting of shell polymer to the core using living polymerization from active sites anchored onto the core polymer; interfacial reaction, i.e., a chemical reaction located at the core particle surface, such as interfacial polycondensation; and using block copolymers as suspending agents during the core particle synthesis.

In some embodiments, the interfacial reaction and use of block polymers are the techniques used when chemical methods are used. In the interfacial reaction pathway, typically, the periphery of the core particle is chemically modified by reacting small molecules or macromolecules on the core interface. For example, an amine containing ion-binding core particle is reacted with a polymer containing amine reactive groups such as epoxy, isocyanate, activated esters, halide groups to form a crosslinked shell around the core.

In another embodiment, the shell is first prepared using interfacial polycondensation or solvent coacervation to produce capsules. The interior of the capsule is then filled up with core-forming precursors to build the core within the shell capsule.

In some embodiments, using the block copolymer approach, an amphiphilic block copolymer can be used as a suspending agent to form the core particle in an inverse or direct suspension particle forming process. When an inverse water-in-oil suspension process is used, then the block copolymer comprises a first block soluble in the continuous oil phase and another hydrophilic block contains functional groups that can react with the core polymer. When added to the aqueous phase, along with core-forming precursor, and the oil phase, the block copolymer locates to the water-in-oil interface and acts as a suspending agent. The hydrophilic block reacts with the core material, or co-reacts with the core-forming precursors. After the particles are isolated from the oil phase, the block copolymers form a thin shell covalently attached to the core surface. The chemical nature and length of the blocks can be varied to vary the permeation characteristics of the shell towards solutes of interest.

When the shell material is physically adsorbed on the core material, well known techniques of microencapsulation such as solvent coacervation, fluidized bed spray coater, or multiemulsion processes can be used. One method of microencapsulation is the fluidized bed spray coater in the Wurster configuration. In yet another embodiment, the shell material is only acting temporarily by delaying the swelling of the core particle while in the mouth and esophagus, and optionally disintegrates in the stomach or duodenum. The shell is then selected in order to hinder the transport of water into the core particle, by creating a layer of high hydrophobicity and very low liquid water permeability.

In one embodiment the shell material carries negative charges while being in the milieu of use. Not being limited to one mechanism of action, it is thought that negatively charged shell material coated on anion-binding beads enhance the binding of small inorganic ions with a low charge density (such as phosphate) over competing ions with greater valency or size. Competing anions such as citrate, bile acids and fatty acids among others, may thus have a lesser relative affinity to the anion binding core possibly as a result of their limited permeability across the shell.

In some embodiments, shell materials are polymers carrying negative charges in the pH range typically found in the intestine. Examples include, but are not limited to, polymers that have pendant acid groups such as carboxylic, sulfonic, hydrosulfonic, sulfamic, phosphoric, hydrophosphoric, phosphonic, hydrophosphonic, phosphoramidic, phenolic, boronic and a combination thereof. The polymer can be protonated or unprotonated; in the latter case the acidic anion can be neutralized with pharmaceutically acceptable cations such as Na, K, Li, Ca, Mg, and NH₄.

In another embodiment the polyanion can be administered as a precursor that ultimately activates as a polyanion: for instance certain labile ester or anhydride forms of either polysulfonic or polycarboxylic acids are prone to hydrolysis in the acidic environment of the stomach and can convert to the active anions.

The shell polymers can be either linear, branched, hyperbranched, segmented (i.e. backbone polymer arranged in sequence of contiguous blocks of which at least one contains pendant acidic groups), comb-shaped, star-shaped or crosslinked in a network, fully and semi-interpenetrated network (IPN). The shell polymers are either random or blocky in composition and either covalently or physically attached to the core material. Examples of such shell polymers include, but are not limited to acrylic acid homopolymers or copolymers, methacrylic acid homopolymers or copolymers, and copolymers of methacrylate and methacrylic acid. Examples of such polymers are copolymers of methylmethacrylate and methacrylic acid and copolymers of ethylacrylate and methacrylic acid, sold under the tradename Eudragit (Rohm GmbH & Co. KG): examples of which include Eudragit L100-55 and Eudragit L100 (a methylmethacrylate-methacrylic acid (1:1) copolymer, Degussa/Rohm), Eudragit L30-D55, Eudragit S 100-55 and Eudragit FS 30D, Eudragit S. 100 (a methylmethacrylate-methacrylic acid (2:1) copolymer), Eudragit LD-55 (an ethylacrylate-methacrylic acid (1:1) copolymer), copolymers of acrylates and methacrylates with quaternary ammonium groups, sold under the tradenames Eudragit RL and Eudragit RS, and a neutral ester dispersion without any functional groups, sold under the tradename Eudragit NE30-D.

Additional shell polymers include: poly(styrene sulfonate), Polycarbophil®; Polyacrylic acid(s); carboxymethyl cellulose, cellulose acetate phthalate, hydroxypropyl methylcellulose phthalate as sold under the tradename HP-50 and HP-55 (Shin-Etsu Chemical Co., Ltd.), cellulose acetate trimellitate, cellulose acetate, cellulose acetate butyrate, cellulose acetate propionate, ethyl cellulose, cellulose derivatives, such as hydroxypropylmethylcellulose, methylcelluose, hydroxylethylcellulose, hydroxyethylmethylcellulose, hydroxylethylethylcelluose and hydroxypropylethylcellulose and cellulose derivatives such as cellulose ethers useful in film coating formulations, polyvinyl acetate phthalate, carrageenan, alginate, or poly(methacrylic acid) esters, acrylic/maleic acid copolymers, styrene/maleic acid polymers, itaconic acid/acrylic copolymers, and fumaric/acrylic acid copolymers, polyvinyl acetal diethylaminoacetate, as sold under the tradename AEA (Sankyo Co., Ltd.), methylvinylether/maleic acid copolymers and shellac.

In some embodiments the shell polymers are selected amongst pharmaceutically acceptable polymers such as Eudragit L100-55 and Eudragit L100 (a methylmethacrylate-methacrylic acid (1:1) copolymer, Degussa/Rohm), Carbopol 934 (polyacrylic acid, Noveon), C-A-P NF (cellulose acetate phthalate—Eastman), Eastacryl (methacrylic acid esters—Eastman), Carrageenan and Alginate (FMC Biopolymer), Anycoat—P (Samsung Fine Chemicals—HPMC Phthalate), or Aqualon (carboxymethyl cellulose—Hercules), methylvinylether/maleic acid copolymers (Gantrez), and styrene/maleic acid (SMA).

The shell can be coated by a variety of methods. In one embodiment, the shell materials are added in the drug formulation step as an active excipient; for example, the shell material can be included in a solid formulation as a powder, which is physically blended with the organophosphate or phosphate-binding polymer and other excipients, optionally granulated, and compressed to form a tablet. Thus, in some embodiments, the shell material need not cover the core material in the drug product. For example, the acidic shell polymer may be added together with the anion binding core polymer formulated in the shape of a tablet, capsule, gel, liquid, etc, wafer, extrudates and the shell polymer can then dissolve and distribute itself uniformly as a shell coating around the core while the drug product equilibrates in the mouth, esophagus or ultimately in the site of action, i.e. the GI tract.

In some embodiments, the shell is a thin layer of shell polymer. The layer can be a molecular layer of polyanion on the core particle surface. The weight to core ratio can be between about 0.0001% to about 30%, preferably comprised between about 0.01% to about 5%, such as between about 0.1% to about 5%.

The shell polymers have a minimum molecular weight such that they do not freely permeate within the core pore volume nor elute from the core surface. In some embodiments, the molecular weight (Mw) of the shell acidic polymer is above about 1000 g/mole, such as above about 5000 g/mole, and or even above about 20,000 g/mole

The anionic charge density of the shell material (as prevailing in the milieu of use) is may be between 0.5 mEq/gr to 22 mEq/gr, such as 2 mEq/gr to 15 mEq/gr. If a coating process is used to form the shell on the polymer particles as part of the manufacture of the dosage form, then procedures known from those skilled-in-the-art in the pharmaceutical industry are applicable. In one embodiment, the shell is formed in a fluidized bed coater (Wurster coater). In an alternate embodiment, the shell is formed through controlled precipitation or coacervation, wherein the polymer particles are suspended in a polymer solution, and the solvent properties are changed in such a way as to induce the polymer to precipitate onto or coat the polymer particles.

Suitable coating processes include the procedures typically used in the pharmaceutical industry. Typically, selection of the coating method is dictated by a number of parameters, that include, but are not limited to the form of the shell material (bulk, solution, emulsion, suspension, melt) as well as the shape and nature of the core material (spherical beads, irregular shaped, etc.), and the amount of shell deposited. In addition, the cores may be coated with one or more shells and may comprise multiple or alternating layers of shells.

The term “phosphate imbalance disorder” as used herein refers to conditions in which the level of phosphorus present in the body is abnormal. One example of a phosphate imbalance disorder includes hyperphosphatemia. The term “hyperphosphatemia” as used herein refers to a condition in which the element phosphorus is present in the body at an elevated level. Typically, a patient is often diagnosed with hyperphosphatemia if the blood phosphate level is, for example, above about 4.0 or 4.5 milligrams per deciliter of blood, for example above about 5.0 mg/dl, such as above about 5.5 mg/dl, for example above 6.0 mg/dl, and/or a severely impaired glomerular filtration rate such as, for example, less than about 20% of normal. The present invention may also be used to treat patients suffering from hyperphosphatemia in End Stage Renal Disease and who are also receiving dialysis treatment (e.g., hemodialysis or peritoneal dialysis).

Other diseases that can be treated with the methods, compounds, compositions, and kits of the present invention include hypocalcemia, hyperparathyroidism, depressed renal synthesis of calcitriol, tetany due to hypocalcemia, renal insufficiency, and ectopic calcification in soft tissues including calcifications in joints, lungs, kidney, conjunctiva, and myocardial tissues. Also, the present invention can be used to treat Chronic Kidney Disease (CKD), End Stage Renal Disease (ESRD) and dialysis patients, including prophylactic treatment of any of the above.

The amine polymers and compositions described herein can be used as an adjunct to other therapies e.g. those employing dietary control of phosphorus intake, dialysis, inorganic metal salts and/or other polymer resins.

The compositions of the present invention are also useful in removing chloride, bicarbonate, oxalate, and bile acids from the gastrointestinal tract. Amine polymers removing oxalate compounds or ions find use in the treatment of oxalate imbalance disorders, such as oxalosis or hyperoxaluria that increases the risk of kidney stone formation. Amine polymers removing chloride compounds or ions find use in treating acidosis, heartburn, acid reflux disease, sour stomach or gastritis, for example. In some embodiments, the compositions of the present invention are useful for removing fatty acids, bilirubin, and related compounds. Some embodiments may also bind and remove high molecular weight molecules like proteins, nucleic acids, vitamins or cell debris.

The present invention provides methods, pharmaceutical compositions, and kits for the treatment of animals. The term “animal” or “animal subject” or “patient” as used herein includes humans as well as other mammals (e.g., in veterinary treatments, such as in the treatment of dogs or cats, or livestock animals such as pigs, goats, cows, horses, chickens and the like). One embodiment of the invention is a method of removing phosphorous-containing compounds such as organophosphates or phosphate from the gastrointestinal tract, such as the stomach, small intestine or large intestine of an animal by administering an effective amount of at least one of the amine polymers described herein.

The term “treating” and its grammatical equivalents as used herein includes' achieving a therapeutic benefit and/or a prophylactic benefit. By therapeutic benefit is meant eradication, amelioration, or prevention of the underlying disorder being treated. For example, in a hyperphosphatemia patient, therapeutic benefit includes eradication or amelioration of the underlying hyperphosphatemia. Also, a therapeutic benefit is achieved with the eradication, amelioration, or prevention of one or more of the physiological symptoms associated with the underlying disorder such that an improvement is observed in the patient, notwithstanding that the patient may still be afflicted with the underlying disorder. For example, administration of amine polymers, described herein, to a patient suffering from renal insufficiency and/or hyperphosphatemia provides therapeutic benefit not only when the patient's serum phosphate level is decreased, but also when an improvement is observed in the patient with respect to other disorders that accompany renal failure and/or hyperphosphatemia like ectopic calcification and renal osteodystrophy. For prophylactic benefit, for example, the amine polymers may be administered to a patient at risk of developing hyperphosphatemia or to a patient reporting one or more of the physiological symptoms of hyperphosphatemia, even though a diagnosis of hyperphosphatemia may not have been made.

The compositions may also be used to control serum phosphate in subjects with elevated phosphate levels, for example, by changing the serum level of phosphate towards a normal or near normal level, for example, towards a level that is within 10% of the normal level of a healthy patient.

Other embodiments of the invention are directed towards pharmaceutical compositions comprising at least one of the amine polymers or a pharmaceutically acceptable salt of the amine polymer, and one or more pharmaceutically acceptable excipients, diluents, or carriers and optionally additional therapeutic agents. The compounds may be lyophilized or dried under vacuum or oven before formulating.

The excipients or carriers are “acceptable” in the sense of being compatible with the other ingredients of the formulation and not deleterious to the recipient thereof. The formulations can conveniently be presented in unit dosage form and can be prepared by any suitable method. The methods typically include the step of bringing into association the agent with the excipients or carriers such as by uniformly and intimately bringing into association the amine polymer with the excipients or carriers and then, if necessary, dividing the product into unit dosages thereof.

The pharmaceutical compositions of the present invention include compositions wherein the amine polymers are present in an effective amount, i.e., in an amount effective to achieve therapeutic and/or prophylactic benefit. The actual amount effective for a particular application will depend on the patient (e.g. age, weight) the condition being treated; and the route of administration.

The dosages of the amine polymers in animals will depend on the disease being, treated, the route of administration, and the physical characteristics of the animal being treated. Such dosage levels in some embodiments for either therapeutic and/or prophylactic uses may be from about 1 gm/day to about 30 gm/day, for example from about 2 gm/day to about 20 gm/day or from about 3 gm/day to about 7 gm/day. The dose of the amine polymers described herein can be less than about 50 gm/day, less than about 40 gm/day, less than about 30 gm/day, less than about 20 gm/day, and less than about 10 gm/day.

Typically, the amine polymers can be administered before or after a meal, or with a meal. As used herein, “before” or “after” a meal is typically within two hours, preferably within one hour, more preferably within thirty minutes, most preferably within ten minutes of commencing or finishing a meal, respectively.

Generally, it is preferred that the amine polymers are administered along with meals. The amine polymers may be administered one time a day, two times a day, or three times a day. Preferably the amine polymers are administered once a day with the largest meal.

Preferably, the amine polymers may be used for therapeutic and/or prophylactic benefits and can be administered alone or in the form of a pharmaceutical composition. The pharmaceutical compositions comprise the amine polymers, one or more pharmaceutically acceptable carriers, diluents or excipients, and optionally additional therapeutic agents. For example, the amine polymers of the present invention may be co-administered with other active pharmaceutical agents depending on the condition being treated. Examples of pharmaceutical agents that may be co-administered include, but are not limited to:

Other phosphate sequestrants including pharmaceutically acceptable lanthanum, calcium, aluminum, magnesium and zinc compounds, such as acetates, carbonates, oxides, hydroxides, citrates, alginates, and ketoacids thereof.

Calcium compounds, including calcium carbonate, acetate (such as PhosLo® calcium acetate tablets), citrate, alginate, and ketoacids, have been utilized for phosphate binding.

Aluminium-based phosphate sequestrants, such as Amphojel® aluminium hydroxide gel, have also been used for treating hyperphosphatemia. These compounds complex with intestinal phosphate to form highly insoluble aluminium phosphate; the bound phosphate is unavailable for absorption by the patient.

The most commonly used lanthanide compound, lanthanum carbonate (Fosrenol®) behaves similarly to calcium carbonate.

Other phosphate sequestrants suitable for use in the present invention include pharmaceutically acceptable magnesium compounds. Various examples of pharmaceutically acceptable magnesium compounds are described in U.S. Provisional Application No. 60/734,593 filed Nov. 8, 2005, the entire teachings of which are incorporated herein by reference. Specific suitable examples include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof.

Various examples of pharmaceutically acceptable zinc compounds are described in PCT Application No. PCT/US2005/047582 filed Dec. 29, 2005, the entire teachings of which are incorporated herein by reference. Specific suitable examples of pharmaceutically acceptable zinc compounds include zinc acetate, zinc bromide, zinc caprylate, zinc carbonate, zinc chloride, zinc citrate, zinc formate, zinc hexafluorosilicate, zinc iodate, zinc iodide, zinc iodide-starch, zinc lactate, zinc nitrate, zinc oleate, zinc oxalate, zinc oxide, calamine (zinc oxide with a small proportion of ferric oxide), zinc p-phenolsulfonate, zinc propionate, zinc salicylate, zinc silicate, zinc stearate, zinc sulfate, zinc sulfide, zinc tannate, zinc tartrate, zinc valerate and zinc ethylenebis(dithiocarbamate). Another example includes poly(zinc acrylate).

When referring to any of the above-mentioned phosphate sequestrants, it is to be understood that mixtures, polymorphs and solvates thereof are encompassed.

In some embodiments, a mixture of the phosphate sequestrants described above can be used in the invention in combination with pharmaceutically acceptable ferrous iron salts.

In other embodiments, the phosphate sequestrant used in combination with compounds of the present invention is not a pharmaceutically acceptable magnesium compound. In yet other embodiments, the phosphate sequestrant used in combination with the pharmaceutically acceptable amine compounds and/or polymers is not a pharmaceutically acceptable zinc compound.

The invention also includes methods and pharmaceutical compositions directed to a combination therapy of the amine polymers in combination with a phosphate transport inhibitor or an alkaline phosphatase inhibitor. Alternatively, a mixture of the amine polymers is employed together with a phosphate transport inhibitor or an alkaline phosphatase inhibitor.

Suitable examples of phosphate transport inhibitors can be found in co-pending U.S. Application Publication Nos. 2004/0019113 and 2004/0019020 and WO 2004/085448, the entire teachings of each of which are incorporated herein by reference.

A large variety of organic and inorganic molecules are inhibitors to alkaline phosphatase (ALP) (see, for example, U.S. Pat. No. 5,948,630, the entire teachings of which are incorporated herein by reference). Examples of alkaline phosphatase inhibitors include orthophosphate, arsenate, L-phenylalanine, L-homoarginine, tetramisole, levamisole, L-p-Bromotetramisole, 5,6-Dihydro-6-(2-naphthyl)imidazo-[2,1-b]thiazole (napthyl) and derivatives thereof. The preferred inhibitors include, but are not limited to, levamisole, bromotetramisole, and 5,6-Dihydro-6-(2-naphthyl)imidazo-[2,1-b]thiazole and derivatives thereof.

This co-administration can include simultaneous administration of the two agents in the same dosage form, simultaneous administration in separate dosage forms, and separate administration. For example, for the treatment of hyperphosphatemia, the amine polymers may be co-administered with calcium salts which are used to treat hypocalcemia resulting from hyperphosphatemia.

The pharmaceutical compositions of the invention can be formulated as a tablet, sachet, slurry, food formulation, troche, capsule, elixir, suspension, syrup, wafer, chewing gum or lozenge.

Preferably, the amine polymers or the pharmaceutical compositions comprising the amine polymers is administered orally. Illustrative of suitable methods, vehicles, excipients and carriers are those described, for example, in Remington's Pharmaceutical Sciences, 19th ed., the contents of which is incorporated herein by reference.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Suitable techniques for preparing pharmaceutical compositions of the amines are well known in the art.

In some aspects of the invention, the amine polymer(s) provide mechanical and thermal properties that are usually performed by excipients, thus decreasing the amount of such excipients required for the formulation. In some embodiments the amine polymer or composition constitutes over about 30 wt. %, for example over about 40 wt. %, over about 50 wt. %, preferably over about 60 wt. %, over about 70 wt. %, more preferably over about 80 wt. %, over about 85 wt. % or over about 90 wt. % of the composition, the remainder comprising suitable excipient(s).

In some embodiments, the compressibility of the tablets is strongly dependent upon the degree of hydration (moisture content) of the amine polymer. Preferably, the amine polymer has a moisture content of about 5% by weight or greater, more preferably, the moisture content is from about 5% to about 9% by weight, and most preferably about 7% by weight. It is to be understood that in embodiments in which the amine polymer is hydrated, the water of hydration is considered to be a component of the amine polymer.

The tablet can further comprise one or more excipients, such as hardeners, glidants and lubricants, which are well known in the art. Suitable excipients include colloidal silicon dioxide, stearic acid, magnesium silicate, calcium silicate, sucrose, calcium stearate, glyceryl behenate, magnesium stearate, talc, zinc stearate and sodium stearylfumarate.

The tablet core of embodiments of the invention may be prepared by a method comprising the steps of: (1) hydrating or drying the amine polymer to the desired moisture level; (2) blending the amine polymer with any excipients; and (3) compressing the blend using conventional tableting technology.

In some embodiments, the invention relates to a stable, swallowable coated tablet, particularly a tablet comprising a hydrophilic core, such as a tablet comprising the amine polymer, as described above. In one embodiment, the coating composition comprises a cellulose derivative and a plasticizing agent. The cellulose derivative is, preferably, hydroxypropylmethylcellulose (HPMC). The cellulose derivative can be present as an aqueous solution. Suitable hydroxypropylmethylcellulose solutions include those containing HPMC low viscosity and/or HPMC high viscosity. Additional suitable cellulose derivatives include cellulose ethers useful in film coating formulations. The plasticizing agent can be, for example, an acetylated monoglyceride such as diacetylated monoglyceride. The coating composition can further include a pigment selected to provide a tablet coating of the desired color. For example, to produce a white coating, a white pigment can be selected, such as titanium dioxide.

In one embodiment, the coated tablet of the invention can be prepared by a method comprising the step of contacting a tablet core of the invention, as described above, with a coating solution comprising a solvent, at least one coating agent dissolved or suspended in the solvent and, optionally, one or more plasticizing agents. Preferably, the solvent is an aqueous solvent, such as water or an aqueous buffer, or a mixed aqueous/organic solvent. Preferred coating agents include cellulose derivatives, such as hydroxypropylmethylcellulose. Typically, the tablet core is contacted with the coating solution until the weight of the tablet core has increased by an amount ranging from about 4% to about 6%, indicating the deposition of a suitable coating on the tablet core to form a coated tablet.

Other pharmaceutical excipients useful in the some compositions of the invention include a binder, such as microcrystalline cellulose, carbopol, providone and xanthan gum; a flavoring agent, such as mannitol, xylitol, maltodextrin, fructose, or sorbitol; a lubricant, such as vegetable based fatty acids; and, optionally, a disintegrant, such as croscarmellose sodium, gellan gum, low-substituted hydroxypropyl ether of cellulose, sodium starch glycolate. Such additives and other suitable ingredients are well-known in the art; see, e.g., Gennaro A R (ed), Remington's Pharmaceutical Sciences, 19th Edition.

In some embodiments the amine polymers of the invention are provided as pharmaceutical compositions in the form of chewable tablets. In addition to the active ingredient, the following types of excipients are commonly used: a sweetening agent to provide the necessary palatability, plus a binder where the former is inadequate in providing sufficient tablet hardness; a lubricant to minimize frictional effects at the die wall and facilitate tablet ejection; and, in some formulations a small amount of a disintegrant is added to facilitate mastication. In general excipient levels in currently-available chewable tablets are on the order of 3-5 fold of active ingredient(s) whereas sweetening agents make up the bulk of the inactive ingredients. In some embodiments the invention provides a pharmaceutical composition formulated as a chewable tablet, comprising an amine polymer described herein, a filler, and a lubricant. In some embodiments the invention provides a pharmaceutical composition formulated as a chewable tablet, comprising an amine polymer described herein, a filler, and a lubricant, wherein the filler is chosen from the group consisting of sucrose, mannitol, xylitol, maltodextrin, fructose, and sorbitol, and wherein the lubricant is a magnesium fatty acid salt, such as magnesium stearate.

In one embodiment, the amine polymer is pre-formulated with a high Tg/high melting point low molecular weight excipient such as mannitol, sorbose, sucrose in order to form a solid solution wherein the polymer and the excipient are intimately mixed. Methods of mixing such as extrusion, spray-drying, chill drying, lyophilization, or wet granulation are useful. Indication of the level of mixing is given by known physical methods such as differential scanning calorimetry or dynamic mechanical analysis.

In some embodiments the amine polymers of the invention are provided as pharmaceutical compositions in the form of liquid formulations. In some embodiments the pharmaceutical composition contains polymer dispersed in a suitable liquid excipient. Suitable liquid excipients are known in the art; see, e.g., Remington's Pharmaceutical Sciences.

In some embodiments, the pharmaceutical compositions may be in the form of a powder formulation packaged as a sachet that may be mixed with water or other ingestible liquid and administered orally as a drink (solution or suspension). In order to ensure that such formulations provide acceptable properties to the patient such as mouth feel and taste, a pharmaceutically acceptable anionic stabilizer may be included in the formulation.

Examples of suitable anionic stabilizers include anionic polymers such as: an anionic polypeptide, an anionic polysaccharide, or a polymer of one or more anionic monomers such as polymers of mannuronic acid, guluronic acid, acrylic acid, methacrylic acid, glucuronic acid glutamic acid or a combination thereof, and pharmaceutically acceptable salts thereof. Other examples of anionic polymers include cellulose, such as carboxyalkyl cellulose or a pharmaceutically acceptable salt thereof. The anionic polymer may be a homopoloymer or copolymer of two or more of the anionic monomers described above. Alternatively, the anionic copolymer may include one or more anionic monomers and one or more neutral comonomers such as olefinic anionic monomers such as vinyl alcohol, acrylamide, and vinyl formamide.

Examples of anionic polymers include alginates (e.g. sodium alginate, potassium alginate, calcium alginate, magnesium alginate, ammonium alginate, and esters of alginate), carboxymethyl cellulose, polylactic acid, polyglutamic acid, pectin, xanthan, carrageenan, furcellaran, gum Arabic, karaya gum, gum ghatti, gum carob, and gum tragacanth. Preferred anionic polymers are alginates and are preferably esterified alginates such as a C2-C5-diol ester of alginate or a C3-C5 triol ester of alginate. As used herein an “esterified alginate” means an alginic acid in which one or more of the carboxyl groups have of the alginic acid are esterified. The remainder of the carboxylic acid groups in the alginate are optionally neutralized (partially or completely) as pharmaceutically acceptable salts. For example, propylene glycol alginate is an ester of alginic acid in which some of the carboxyl groups are esterified with propylene glycol, and the remainder of the carboxylic acid groups are optionally neutralized with pharmaceutically acceptable salts. More preferably, the anionic polymer is ethylene glycol alginate, propylene glycol alginate or glycerol alginate, with propylene glycol alginate even more preferred.

All publications and patent applications mentioned in this specification are herein incorporated by reference to the same extent as if each individual publication or patent application was specifically and individually indicated to be incorporated by reference.

It will be apparent to one of ordinary skill in the art that many changes and modification can be made to the disclosures presented herein without departing from the spirit or scope of the appended claims.

### EXAMPLES

As used herein, the following terms have the meanings ascribed to them unless specified otherwise:

ISCO/Combi-Flash—automated flash chromatography system;

DMAP—N,N-dimethylaminopyridine, commercially available from Aldrich;

HPLC—high performance liquid chromatography;

LC/MS—liquid chromatography/mass spectroscopy; and

Triton® B—trimethylbenzylammonium hydroxide, commercially available from Aldrich.

### Materials Used

Pentaerythritol, dichloromethane (DCM), ethanol, methanol, 1,4-dioxane, D-sorbitol and epichlorohydrin are commercially available from Sigma-Aldrich, Co. and were used without further purification.

Acrylonitrile, commercially available from either Sigma-Aldrich or Alfa Aesar, A Johnson Matthey Company, and was used without further purification.

Raney cobalt was obtained from Aldrich, and was either used as a wet slurry, or was azeotropically dried before use.

### Analytical Techniques

Proton NMR spectra were recorded at 400 MHz on a Varian NMR spectrometer in deuterated chloroform with TMS as an internal standard, unless otherwise indicated. ¹³C NMR experiments were performed on the same instrument, operating at a frequency of 66 MHz.

LC/MS experiments were performed on a Waters Ion Trap LC/MS equipped with a reversed phase Zorbax C-8 column. Samples were eluted with gradient mixtures of acetonitrile:water:formic acid. Ionization was performed using an electrospray source, with the ionization potential set to 30 V.

HPLC measurements were conducted on Agilent instruments, equipped with a Zorbax C-8 column, and an evaporative light scattering detector.

### Example 1

### Synthesis of Compound I

A 101 g sample of pentaerythritol was charged to a 2 L 3-necked round bottom flask under N₂, and was slurried in 500 mL of acrylonitrile and 500 mL of 1,4-dioxane. A 9 mL portion of 40% KOH solution, and 18 mL of water were added to the reaction mixture, and the mixture stirred at room temperature. The reaction was heated to 40° C., at which point the pentaerythritol began dissolving. A slow exotherm began, and the reaction was cooled with ice to keep the temperature under 60° C. The reaction was stirred at room temperature overnight, and was analyzed by HPLC the following morning. The reaction mixture was transferred to a large separatory funnel, and was diluted with 2 L of tert-butyl methyl ether. The organic phase was then washed twice with 50% brine, was dried over anhydrous sodium sulfate, was filtered, and was concentrated in vacuo to yield 250 g of a light yellow oil, that solidified upon standing. The material was suitably pure to use for subsequent steps without further purification.

¹H NMR (300 MHz, CDCl₃): δ (ppm) 2.5 (t, 8H); 3.4 (s, 8H); 3.6 (t, 8H).

¹³C NMR (66 MHz, CDCl₃): δ (ppm) 19.019 (CH₂CN); 45.802 (quaternary C); 65.842 (O—CH₂—CH₂); 68.909 (C—CH₂—O); 118.532 (—CN).

HPLC purity (ELSD): >95 AUC.

### Example 2A

### Synthesis of Compound II

A 6 g sample of Compound I was placed in a Parr hydrogenation apparatus, and was suspended in 150 mL of 1:1 MeOH:H₂O. 12 g of wet Raney cobalt catalyst were charged to this mixture, and the reaction vessel sealed. The resulting mixture was hydrogenated under 700 psi H₂ at 70° C. for 18 h. The reaction vessel was cooled to room temperature, the resulting material was analyzed by LC/MS and filtered through a bed of celite. The filtrate was concentrated in vacuo to yield 5.8 g of the desired product as a pale yellow oil.

¹H NMR (300 MHz, D₂O): δ (ppm) 1.7 (m, 8H); 2.5 (t, 8H); 3.2 (s, 8H); 3.4 (t, 8H).

HPLC purity (ELSD): >98% AUC.

LC/MS [M+H]⁺ m/z=365.5 (exact mass of compound=364.300).

### Example 2B

### Synthesis of Compound II

A 50 g sample of Compound I was placed in a Parr hydrogenation apparatus. To this, 5 g of freshly dried Raney cobalt was added in 30 mL of toluene, under N₂. The hydrogenation apparatus was sealed, and evacuated. 20 psi of anhydrous ammonia was introduced, followed by 1200 psi of hydrogen. The reaction mixture was then heated to 109° C., and was stirred for 12 hr at which point the resulting material was cooled to room temperature and analyzed by LC/MS before being filtered over a small amount of celite (under N₂), with the celite being washed several times with DCM. The filtrate was concentrated in vacuo to give 52 g of the desired product as a yellow oil.

¹H NMR (300 MHz, D₂O): δ (ppm) 1.7 (m, 8H); 2.5 (t, 8H); 3.2 (s, 8H); 3.4 (t, 8H).

HPLC purity (ELSD): >98% AUC.

LC/MS [M+H]⁺ m/z=365.5 (exact mass of compound=364.300).

### Example 3

### Synthesis of Compound III

A 52 g sample of Compound II was charged to a Parr hydrogenation apparatus, along with 112 mL of acylonitrile. The reaction vessel was sealed, and was evacuated. The vessel was then pressurized with 50 psi of N₂, and was heated at 140° C. for 12 h. The reaction vessel was cooled to room temperature and analyzed via HPLC. The reaction mixture was concentrated in vacuo to give˜200 g of crude material. 40 g of this material were purified over normal phase silica gel (0-100% ethyl acetate:hexane mobile phase) to give 28 g of the desired product.

HPLC purity (ELSD): >95% AUC. LC/MS [M+H]⁺ m/z=789.6 (exact mass of compound=788.52).

### Example 4A

### Synthesis of Compound IV

A 3 g sample of Compound III was charged to a Parr hydrogenation apparatus, and was dissolved in a mixture of 150 mL methanol and 50 mL water. 12 g of wet Raney cobalt were added to the reaction vessel. The vessel was sealed, and the reaction hydrogenated at 80° C. under 1500 psi H₂ for 4 days. The reaction was cooled to room temperature and the analyzed via HPLC and LC/MS, filtered over a bed of celite, with the resulting light blue filtrate concentrated under reduced pressure. The resulting oil was suspended in a 1:1 mixture of methanol and DCM, and was dried over anhydrous sodium sulfate and then treated by excess ammonia in methanol. The resulting material was filtered over celite, and the filtrate concentrated in vacuo to yield 3 g of the desired product as a clear oil.

HPLC purity (ELSD): >99% AUC.

### Example 4B

### Synthesis of Compound IV

A 28 g sample of Compound III was charged to a Parr hydrogenation apparatus, along with 10 g of azeotropically dried Raney cobalt in 40 mL of toluene, under N₂. The reaction vessel was sealed, and evacuated. 40 psi of anhydrous ammonia were introduced, followed by 1600 psi H₂. The reaction was hydrogenated at 120° C. for 3 days, at which point it was cooled to room temperature, the resulting material analyzed by HPLC and LC/MS and filtered over celite (under N₂), with the filter pad being washed with several portions of DCM. The filtrate was concentrated in vacuo to yield 26 g of the desired product as a yellow oil. HPLC Purity (ELSD): >95 AUC. LC/MS [M+H]⁺ m/z=822.3 (molecular weight of compound=821.28, exact mass=820.77).

### Example 5

### Reaction of Compound IV with Epichlorohydrin

To a round bottomed flask was added 6.1 g of Compound IV, 6.1 ml of water, and 420 μl of epichlorohydrin. The resulting solution was stirred at room temperature for 1.5 hours, before being heated to 60° C. for 14 hours. The resulting solids were suspended in 1 L of water, and stirred 1 hour. At this time, the suspension had a conductivity of 319 μS, and a pH of 10.5. The suspension pH was then adjusted to 7 with HCl. The resultant gel was then filtered to give 119 g of polymer (swelling index=20). The product was dried at 65° C. in an oven with a constant nitrogen stream for 18 hours, which afforded 2.8 g of a sticky solid. The material was re-swelled in water, and the pH was adjusted to 2 [using HCl]. It was then filtered and dried again. After drying for 3 days, a hygroscopic solid was obtained.

### Example 6

### Synthesis of Compound V

To a round bottom flask was added 36.4 g of D-sorbitol, 200 ml of 1,4-dioxane and 106 ml of acrylonitrile. The resulting solution was cooled to 5° C. on ice, to which was dropwise added a solution of Triton® B (5 ml in 50 mL of dioxane) via addition funnel. The reaction mixture was stirred at room temperature for 18 hours, and was then concentrated under reduced pressure. The resulting residue was taken up in DCM and transferred to a separatory funnel. The organic layer was washed twice with 50% brine. The brine layers were combined and further extracted with DCM. The DCM fractions were combined, dried over anhydrous sodium sulfate, filtered, and then concentrated in vacuo. The resulting residue was purified by flash chromatography over silica gel (0-->90% ethyl acetate in hexanes as a mobile phase) to afford the desired product (55 g) as a light yellow oil.

### Example 7

### Amine Polymer Urinary Phosphorous Reduction (In-vivo Rats)

Reduction of urinary phosphorous of Compound IV crosslinked with 12 wt. % epichlorohydrin (based on the total weight of the crosslinked Compound) was compared to a cellulose control and to Sevelamer according to the method described in the test methods in two studies. Table I details the doses studied and the results obtained.

TABLE I

| | Dose of Test | 24 Hour Urine | % Reduction | 24 hour Urine | % Reduction in |
|---|---|---|---|---|---|
| | Article in Feed | Phosphorous | in Urinary | Phosphorous | Urinary |
| | (% by weight in | (mg/24 hours) | Phosphorous | (mg/24 hours) | Phosphorous |
| Test Article | feed) | Study #1 | Study #1 | Study # 2 | Study #2 |
| Cellulose | 0.50% | 21.4 ± 4.6 | NA | 21.1 ± 5.5 | NA |
| Sevelamer | 0.50% | 13.6 ± 4.7 | 36.4% | 9.3 ± 3.1 | 56.0% |
| 12% | 0.25% | 14.4 ± 1.9 | 32.7% | 13.1 ± 2.0 | 38.0% |
| Epichlorhydrin- | | | | | |
| crosslinked | | | | | |
| Compound IV | | | | | |

### Test Methods

### Amine Polymer Urinary Phosphorous Reduction (In Vivo-Rats)

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats were distributed into groups.

Purina 5002 was used as the standard diet. The amine polymer being tested was mixed with Purina 5002 to result in a final amine polymer concentration of 0.25% by weight of the feed. Cellulose at 0.5% by weight was used as a negative control. Sevelamer at 0.5% by weight was used as a positive control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated. Percentage reduction of urinary phosphorous was determined by the following equation:

$$\% Reduction of Urinary Phosphorous = [ ( phosphorous of negative control ( mg \text{/} day ) - urinary phosphorous of experimental ( mg \text{/} day ) ) / urinary phosphorous of negative control ( mg \text{/} day ) ] \times 100.$$

Vitro Phosphate Binding (mmol/g)

Two samples per polymer are weighed into plastic bottles after having adjusted the weight of the polymer for the loss on drying of each sample. A 10 mM phosphate buffer solution containing 10 mM KH₂PO₄, 100 mM N,N-bis[2-hydroxyethyl]-2-aminoethanesulfonic acid, 80 mM NaCl, 15 mM glycochenodeoxycholic acid (GCDC), and 15 mM oleic acid (pH adjusted to 7.0 with 1 N NaOH) is prepared and well mixed. Aliquots of the 10 mM phosphate buffer solution is transferred into each of the two sample bottles. The solutions are well mixed and then placed into an orbital shaker at 37° C. for 1 hour. The polymer is allowed to settle prior to removing a sample aliquot from each solution. The sample aliquot is filtered into a small vial using a disposable syringe and syringe filter. The filtered sample is diluted 1-to-10 with DI water. The shaking is continued for a further 4 hours (total of 5 hours) and the sampling procedure is repeated. Phosphate standards are prepared from a 10 mM phosphate standard stock solution and diluted appropriately to provide standards in the range of 0.3 to 1.0 mM. Both the standards and samples are analyzed by ion chromatography. A standard curve is set up and the unbound phosphate (mM) for each test solution is calculated. Bound phosphate is determined by the following equation:

Bound Phosphate (mmol/g)=[(10−Unbound PO₄)×Vol.×1000]/MassP; wherein

Vol.=volume of test solution (L); MassP=LOD adjusted mass of polymer (mg).

In-Process Swelling Ratio (mL/g)

The in-process swelling ratio (SR) of several examples is determined by the following equation:

SR=(weight of wet gel (g)−weight of dry polymer (g))/weight of dry polymer (g).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-96. (canceled)

97. A pharmaceutical composition comprising:
a) at least one amine polymer comprising:
at least one amine compound or residue thereof wherein the amine compound is represented by the following Formula I: 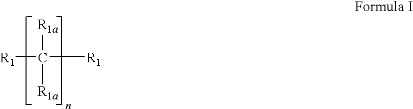
wherein
n independently represents an integer from 1-20;
R₁ independently represents a hydrogen radical, a hydroxyl radical or —OR₃;
R₁ₐ independently represents R₁, —R₂OH or —R₂OR₃; with the proviso that said amine compound comprises at least one moiety represented by R₃;
R₂ independently represents a substituted or un-substituted alkyl radical; and
R₃ independently represents a group represented by the following Formula II: 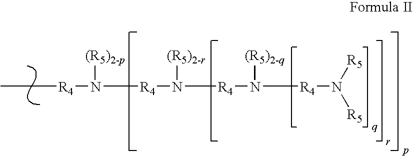
wherein
p, q and r independently represent an integer from 0-2;
R₄ independently represents 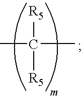
wherein
m independently represents an integer from 1-20;
R₅ independently represents a hydrogen radical; a substituted or un-substituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof;
b) a crosslinking agent or residue thereof; and
c) a pharmaceutically acceptable excipient.

98. The composition of claim 97, wherein the amine compound is represented by the following Formula III:

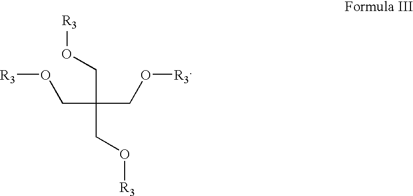

99. The composition of claim 98, wherein the amine compound is represented by the following Formula IV:

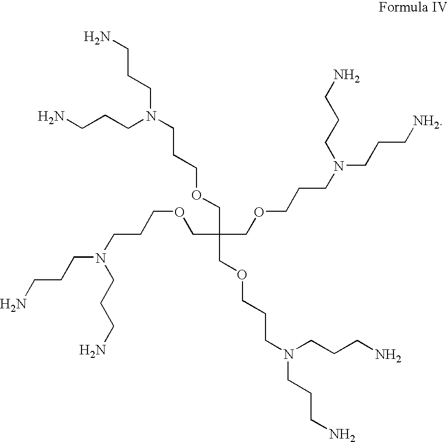

100. The composition of claim 97, wherein the amine compound is represented by the following Formula V:

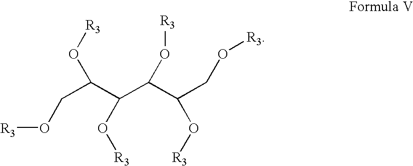

101. The composition of claim 100, wherein the amine compound is represented by the following Formula VI:

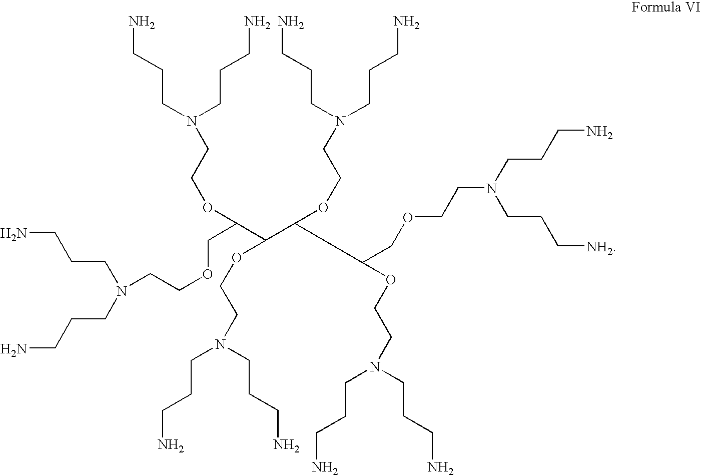

102. The composition of claim 97, wherein the amine compound or residue thereof is derived from erythrose, threose, ribose, arabinose, xylose, lyxose, allose, altrose, glucose, mannose, gulose, idose, galactose, talose, sedoheptulose, sorbose, pentaerythrose, or a partially or fully hydrogenated derivative thereof, or a combination thereof.

103. A method of treating hyperphosphatemia comprising administering to a patient in need thereof a therapeutically effective amount of an amine polymer comprising:
a) at least one amine compound or residue thereof wherein the amine compound is represented by the following Formula I: 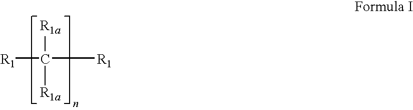
wherein
n independently represents an integer from 1-20;
R₁ independently represents a hydrogen radical, a hydroxyl radical or —OR₃;
R₁ₐ independently represents R₁, —R₂OH or —R₂OR₃; with the proviso that said amine compound comprises at least one moiety represented by R₃;
R₂ independently represents a substituted or un-substituted alkyl radical; and
R₃ independently represents a group represented by the following Formula II: 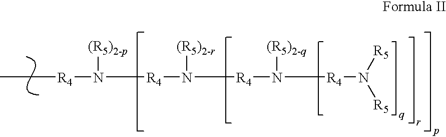
wherein
p, q and r independently represent an integer from 0-2;
R₄ independently represents 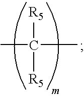
wherein
m independently represents an integer from 1-20;
R₅ independently represents a hydrogen radical; a substituted or un-substituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; and
b) a crosslinking agent or residue thereof.

104. The method of claim 103, wherein the amine compound is represented by the following Formula III:

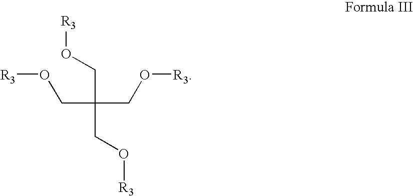

105. The method of claim 104, wherein the amine compound is represented by the following Formula IV:

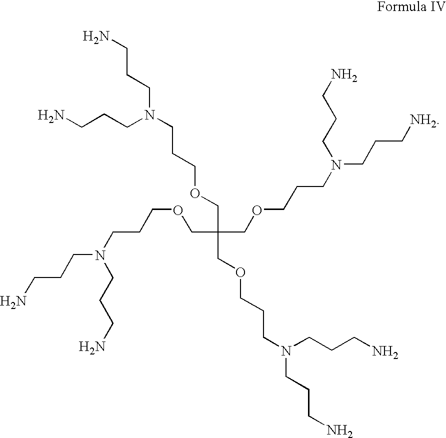

106. The method of claim 103, wherein the amine compound is represented by the following Formula V:

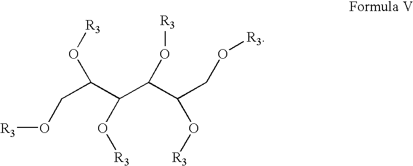

107. The method of claim 106, wherein the amine compound is represented by the following Formula VI:

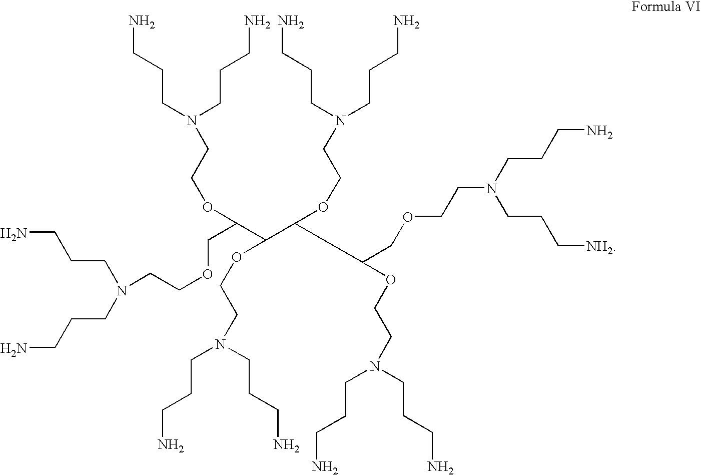

108. The method of claim 103, wherein the amine compound or residue thereof is derived from erythrose, threose, ribose, arabinose, xylose, lyxose, allose, altrose, glucose, mannose, gulose, idose, galactose, talose, sedoheptulose, sorbose, pentaerythrose, or a partially or fully hydrogenated derivative thereof, or a combination thereof.

109. A method of treating hyperphosphatemia comprising administering to a patient in need thereof a therapeutically effective amount of an amine polymer comprising:
a) an amine dendrimer comprising:
i) a core comprising a residue of one or more sugar alcohols; and
ii) a residue of one or more substituted or un-substituted α, β unsaturated nitriles; and
b) a crosslinking agent or residue thereof.

110. A method of treating hyperphosphatemia comprising administering to a patient in need thereof a therapeutically effective amount of an amine polymer comprising
a) an amine compound or residue thereof comprising:
one or more substituted sugar alcohols, wherein one or more of the substitutions includes a group represented by the following Formula XXXIX: 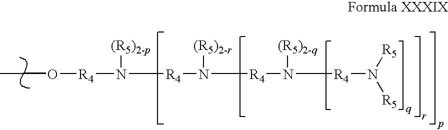
wherein
p, q and r independently represent an integer from 0-2;
R₄ independently represents 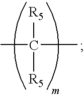
wherein
m independently represents an integer from 1-20;
R₅ independently represents a hydrogen radical; a substituted or un-substituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; and
b) a crosslinking agent or residue thereof.

115. A method of treating hyperphosphatemia comprising administering to a patient in need thereof a therapeutically effective amount of a polymer network comprising:
a) a plurality of units represented by the following Formula XL: 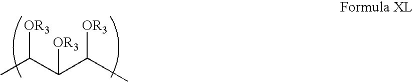
wherein R₃ independently represents a group represented by the following Formula II: 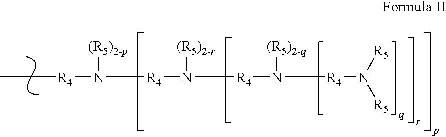
wherein
p, q and r independently represent an integer from 0-2;
R₄ independently represents 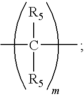
wherein
m independently represents an integer from 1-20;
R₅ independently represents a hydrogen radical; a substituted or un-substituted alkyl radical; a substituted or un-substituted aryl radical; or
R₅ and a neighboring R₅ together represent a link or links comprising a residue of a crosslinking agent, a substituted or un-substituted alicyclic radical, a substituted or un-substituted aromatic radical, or a substituted or un-substituted heterocyclic radical; or R₅ represents a link with another compound or a residue thereof; and
b) a plurality of units represented by the following Formula XLI: [chemical structure]
